(19) United States
(12) Patent Application Publication
(10) Pub. No.: US 2026/0295303 A1
(43) Pub. Date: Oct. 1, 2026

(54) MACHINE LEARNING APPROACH FOR ADAPTIVE RADIATION THERAPY USING COMPOSITE IMAGING SLICES

(71) Applicant: Elekta LTD., Montreal (CA)

(72) Inventors: Francois Paul George Rene Hebert, Montreal (CA); Sebastien Tremblay, Saint-Jean-sur-Richelieu (CA)

(21) Appl. No.: 19/479,323

(22) PCT Filed: May 3, 2024
(86) PCT No.: PCT/CA2024/050607

§ 371 Date: Oct. 27, 2025

(30) Foreign Application Priority Data
May 5, 2023 (GB) 2306699.6

(51) Int. Cl. A61N 5/10

(52) U.S. Cl. CPC A61N 5/1067; A61N 5/1037; A61N 5/1049

(57) ABSTRACT

A radiation therapy treatment plan can be adjusted concurrently with delivery of the therapy in order to compensate for changes in a position of a target. For example, at least two image slices, taken from different in-plane directions, can be used to estimate motion in a through-plane direction substantially orthogonal to the in-plane directions. A machine learning model can be trained with interpolations from a sequence of in-plane image slices, the motion estimated therefrom in the through-plane direction, or both to predict 3D motion.

### CLAIM OF PRIORITY

This application claims priority to UK Patent Application Serial No. GB 2306699.6, filed on May 5, 2023, which is incorporated by reference herein in its entirety, and the benefit of priority of which is claimed herein.

### TECHNICAL FIELD

This disclosure generally relates to radiation therapy or radiotherapy. More specifically, the disclosure relates to systems and methods for adapting a radiation therapy treatment plan such as to compensate for changes in a position of a target tumor and a radiation source with respect to each other during delivery of radiation therapy.

### BACKGROUND

Radiation therapy or “radiotherapy” can be used to treat cancers or other ailments in mammalian (e.g., human and animal) tissue. One such radiotherapy technique is a Gamma Knife, by which a patient is irradiated by a large number of low-intensity gamma rays that converge with high intensity and high precision at a target (e.g., a tumor). In another example, radiotherapy is provided using a linear accelerator, whereby a tumor is irradiated by high-energy particles (e.g., electrons, protons, ions, or high-energy photons). Such an approach involves accurate placement and dosage of the radiation beam, such as to ensure the tumor receives the prescribed radiation and to help minimize damage to the surrounding healthy tissue (e.g., organ(s) at risk (OARs)). Radiation is termed “prescribed” because a physician orders a predefined amount of radiation to the tumor and surrounding organs similar to a prescription for medicine.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various examples discussed in the present document.

FIG. 1A schematically depicts an example of a machine learning approach to motion tracking during radiation treatment.

FIG. 1B schematically depicts an example of a machine learning approach to motion tracking during radiation treatment.

FIG. 2 illustrates generally a view of an object or target locus to be treated using radiation therapy.

FIG. 3A illustrates a view of an example of an imaging acquisition plane of a target locus.

FIG. 3B illustrates a view of an example of an acquired imaging slice corresponding to a first imaging plane orientation intersecting the target locus.

FIG. 3C illustrates a view of an imaging acquisition plane intersecting the target locus along a line, corresponding to a second imaging plane orientation.

FIG. 3D illustrates a view of an acquired imaging slice including a portion of the target locus.

FIG. 3E illustrates a view of an example of an imaging acquisition plane intersecting the target locus.

FIG. 3F illustrates a view of an example of an imaging acquisition plane intersecting the target locus.

FIG. 4A illustrates an example of a series of imaging acquisition planes.

FIG. 4B illustrates an example of successively acquired imaging slices.

FIG. 4C illustrates an example of a series of imaging acquisition planes.

FIG. 5A illustrates an example of a series of two imaging slices.

FIG. 5B illustrates an example of a spatially registered slice.

FIG. 6 illustrates an example of a technique for spatially registering segmented portions of a target locus from imaging slices.

FIG. 7A illustrates an example of a composite representation of a target locus including spatial registration of segmented portions.

FIG. 7B illustrates an example of a technique for determining a difference between a later estimated or predicted location of a composite of the target locus as compared to an earlier location of the target locus.

FIG. 7C illustrates an example of a technique for updating a therapy protocol to shift a therapy locus to a new location based on determined motion.

FIG. 8 illustrates an example of a machine learning engine 800 for use in estimating or predicting motion of a target.

FIG. 9 is a flowchart that describes a method of using an adaptive image-guided therapy delivery system.

FIG. 10A illustrates generally an example of a radiation therapy system.

FIG. 10B illustrates generally another example of a radiation therapy system.

FIG. 11 illustrates generally an example of a system that can include a combined radiation therapy system and an imaging system.

FIG. 12 illustrates generally a partially cut-away view of an example of a system that can include a combined radiation therapy system and an imaging system.

FIG. 13 illustrates generally an example of a multi-leaf collimator.

FIG. 14 illustrates an exemplary radiotherapy system.

### DETAILED DESCRIPTION

The present inventors have recognized, among other things, that a radiation therapy treatment plan can be adjusted concurrently with delivery of the therapy in order to compensate for changes in a position of an anatomical target (e.g., a tumor), OARs, or the radiation source during the delivery of radiation therapy to the tumor. For example, at least two cine image “slices”, taken from different in-plane directions, can be used to estimate motion in a through-plane direction orthogonal to the in-plane directions. In an example, a machine learning model can be trained with interpolations from a sequence of two or less, noncoplanar cine image slices, the motion or position estimated therefrom in the through-plane direction to estimate a real-time three dimensional (3D) position of an anatomical structure. Such a technique can facilitate 3D motion tracking of a tumor without requiring actual monitoring of the tumor via image slices taken in three different directions. In an example, such a technique can even allow for noncoplanar cine image slices, taken in two different directions, to not be orthogonal to each other (e.g., to not be strictly orthogonal sagittal and coronal slices with respect to each other). Such a technique can further allow for 3D motion tracking of a tumor with a single image slice.

Such an approach can help mitigate challenges in radiation therapy caused by undesired motion or misalignment between a target and the radiation source. For example, the tumor can shrink, can move, or can be misaligned compared to the expected or desired location as indicated in the treatment plan. Motion of the target can be caused by one or more sources, such as heart motion, respiration, a reflex such as a cough, or other movements. In other approaches, the source of radiation can be moved, such as intentionally, with respect to the target during the treatment. Therefore, the position of where radiation therapy is to be delivered based on images taken prior to treatment can be significantly misaligned with the desired target when the radiation therapy is eventually delivered.

In one approach, imaging can be performed concurrently with the delivery of a radiation therapy, such as performing an imaging acquisition or motion prediction immediately before initiating radiation therapy delivery during a treatment session, or using a sequence of respective therapy delivery followed by immediately acquiring one or more images of the tumor during a radiation therapy delivery session. Such imaging can provide information helpful for identifying a position of the target or for identifying or predicting the motion of the target. Such contemporaneous imaging can be referred to generically as “online” or “real-time,” but in general a latency or time delay exists between an acquisition of an image and a delivery of radiation therapy, which is generally on the order of about 900 to 1400 milliseconds (ms). In an example, such delay can be reduced such as using machine-learning assisted motion prediction approaches described herein.

Generally, this disclosure focuses on an approach that uses approximately sagittal and coronal 2D cine image slices that are centered at or near the target centroid. Herein, the terms “sagittal” and “coronal” need not refer to orientations strictly orthogonal or perpendicular to each other. Instead, “sagittal” and “coronal” can more loosely describe to two different anatomical directions at which cine images are taken, without being strictly limited to an exact anatomical placement or an exact relationship with each other. In an example, an adaptive image-guided therapy delivery system can receive imaging information including a volumetric image comprising a target within a radiation therapy patient, and can receive imaging information corresponding to one or more imaging slices comprising different portions of the target, the imaging slices acquired at different instants after acquisition of the volumetric image. Also, an adaptive image-guided therapy delivery system can include receiving estimated motion in the through-plane direction, an estimated 3D position of an anatomical target, or both.

Motion tracking can be important in delivery of radiation therapy, such as using a multi-leaf collimator (MLC) approach. In one approach, ionizing radiation in the form of a collimated beam can be directed from an external radiation source toward a patient. A specified or selectable beam energy can be used, such as for delivering a diagnostic energy level range or a therapeutic energy level range. Modulation of a radiation beam can be provided by one or more attenuators or collimators (e.g., an MLC). The intensity and shape of the radiation beam can be adjusted by collimation to avoid damaging healthy tissue (e.g., organs at risk) adjacent to the targeted tissue by conforming the projected beam to a profile of the targeted tissue. Here, motion tracking can be used to track a marker attached to a patient or a body part and to measure changes in the location of a body part such as a tumor or a target organ. Marker and target tracking can be used to deliver the radiosurgery dose, such as to modify leaves of the MLC to shape the beam for desired delivery of radiation. However, the accuracy and reliability of radiation therapy is sensitive to patient movement. When the patient moves, the target or marker moves with the patient and can degrade the accuracy of the radiation dose delivery.

The patient may move because of involuntary muscle contractions or voluntary muscle movements. For example, movement of the patient during radiation therapy can be caused by breathing in a normal or abnormal manner or as a result of disease or injury that causes the patient to breathe unexpectedly. In addition, for some patients it is undesirable to position the body part undergoing radiation therapy in a stable position for an extended period of time, such as when the patient is conscious. In some instances, patient positioning and stabilization systems can be used to help reduce patient movement.

Generally, radiation therapy involves accurate dosing to achieve an effective treatment. For example, radiation can be delivered in one or more fractions, such as fractions of a daily dose. To ensure that a fraction of a daily dose is delivered to the patient without error, the total treatment dose can be delivered in a number of separate short treatment sessions. Here, a daily radiation dose can be further subdivided into many short fractions to reduce the patient's exposure to radiation. When the treatment schedule is a daily schedule, patient motion that occurs between treatment sessions can degrade the effectiveness of the radiation treatment and the patient may be subjected to a larger or smaller radiation dose, than would be incurred if the patient did not move between treatments.

A treatment planning procedure can be used such as to determine a dosage of radiation to deliver around a target area. For example, a treatment planning procedure can involve using cine images of the patient to identify a target region (e.g., the tumor) and to identify critical organs near the tumor. Creation of a treatment plan can be a time-consuming process where a planner tries to comply with various treatment objectives or constraints (e.g., dose volume histogram (DVH) objectives), accounting for their individual importance (e.g., weighting) such as to produce an effective treatment plan. In an approach to treatment planning, for each patient, the initial treatment plan can be generated in an “offline” manner. The treatment plan can be developed well before radiation therapy is delivered, such as using one or more medical imaging techniques. Imaging information can include, for example, images from X-rays, Computed Tomography (CT), nuclear magnetic resonance (MR), positron emission tomography (PET), single-photon emission computed tomography (SPECT), or ultrasound. A health care provider, such as a physician, can use three-dimensional (3D) imaging information indicative of the patient anatomy to identify one or more target tumors along with the organs at risk near the tumor. The health care provider can delineate the target tumor that is to receive a prescribed radiation dose using a manual technique, and the health care provider can similarly delineate nearby tissue, such as organs, at risk of damage from the radiation treatment. The initial treatment plan can be adapted to the 3D pre-treatment image to compensate for any anatomical changes and set-up differences. During radiation treatment, the target can be tracked, and therapy can be paused if the target moves outside a specified margin, such as to avoid undesired or insufficient delivery of radiation. A challenge with this approach is that it involves delay of the procedure and manual repositioning or alignment of the radiation source with respect to the patient.

The present systems and techniques solve the technical problem of online motion tracking by concurrently updating a radiation treatment plan during the treatment. Such a “real-time” motion tracking technique can help compensate for movement of the patient or the equipment during a procedure and can reduce a need to pause and manually realign in the instance of such movement. For example, during radiation treatment, additional two-dimensional (2D) cine image slices can be acquired and used to establish or adjust the beam based on determined or predicted motion of the target. A machine learning model can be actively trained during treatment such as to estimate motion in the through-plane direction, without obtaining a cine image in the through-plane direction, using two or less cine image slices from an in-plane direction. The machine learning model can also be trained such as to estimate a 3D position of a target (e.g., including a through-plane coordinate), using two or less cine image slices from a through-plane direction. In an example, the machine learning model can be used to predict motion in the in-plane direction or the through-plane direction. Such tracking and prediction of motion can be incorporated, concurrent with therapy delivery, into a dynamic treatment plan to mitigate undesired radiation dose delivery resulting from a moving target.

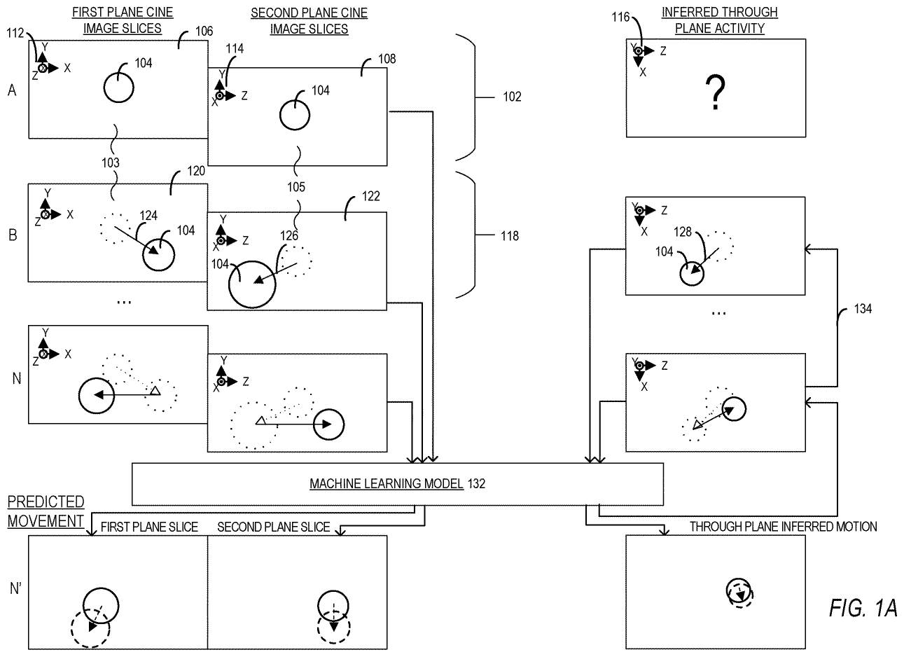

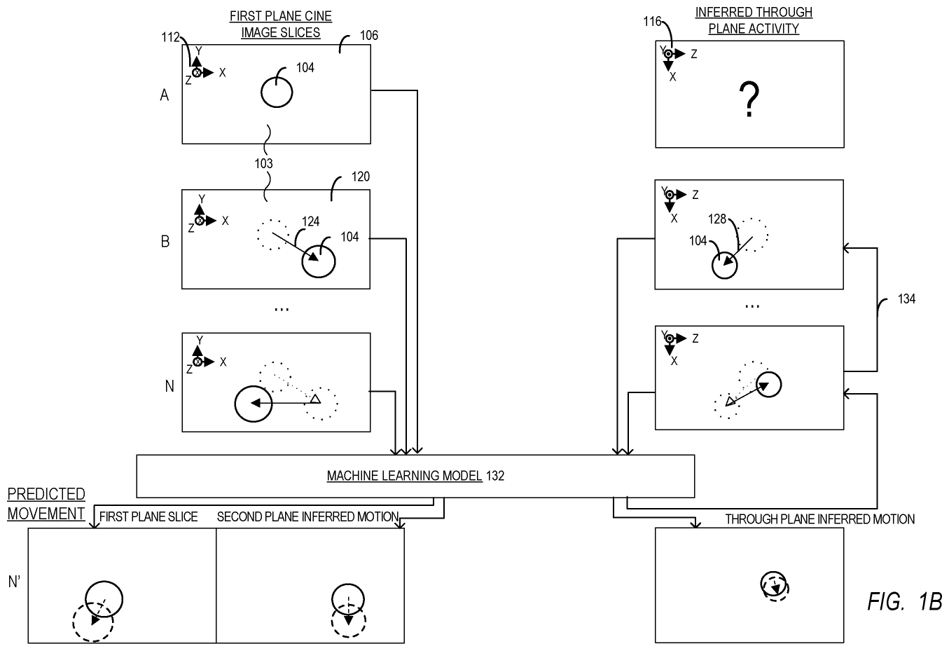

FIG. 1A and FIG. 1B schematically depict examples of a machine learning approach to motion tracking during radiation treatment. At or near time point A, an adaptive image-guided therapy delivery system can receive, during a radiation session involving delivery of radiation to a patient, a first image pair 102. The first image pair 102 can correspond with a specified anatomical structure 104 of the patient. The first image pair can include at least two first image slices, such as an approximately coronal image slice 106 and an approximately sagittal image slice 108 of the structure 104 at two respective different planes. The two respective different planes need not necessarily be orthogonal to each other (e.g., not strictly or literally “sagittal” and “coronal”) and instead can be any two anatomical planes that are not coplanar with each other. While described herein as an image “pair” 102, the image slices 106 and 108 need not be actually correlated to or in any way grouped with each other by the adaptive image-guided therapy system and can be handled by the system independently from each other. Also, the at least two respective planes can form an angle relative to each other, wherein the angle can be a pre-determined angle determined based on anatomical information corresponding to the patient. For example, the pre-determined angle can be between about 45° and 135°, between about 30° and 90°, or between about 60° and 90°. The pre-determined angle can be established as near 90° such as to help mitigate unwanted errors or decay of through-plane estimation accuracy, such as errors and decay arising when the pre-determined angle approaches 0° (parallel). In another example, the at two respective different planes can be oriented substantially orthogonal to each other. Herein, “substantially orthogonal” can refer to angles that are included in a range of ±10° of an orthogonal angle. In an example, the at least two first image slices can be captured at different points in time from each other, such as points of time within a time frame. The different points in time can be described herein and represented in FIG. 1A as taken at approximately the same time point A, as one another, while still being understood as distinguishable, different points in time. Also, the adaptive image-guided therapy delivery system can receive the first image pair 102, including in-plane cine image slices (e.g., a first plane 112 and a second plane 114) within the time frame without receiving any through-plane cine image slices (e.g., a third plane 116) at about the same time point A.

Subsequently, at or near time point B, the adaptive image-guided therapy delivery system can receive, during the radiation session, a second image pair 118. The second image pair can be similar to the first image pair, such as including at least two second image slices, such as a coronal image slice 120 and sagittal image slice 122 slices. While described herein as an image “pair” 118, the image slices 120 and 122 need not be actually correlated to or in any way grouped with each other by the adaptive image-guided therapy system and can be handled by the system independently from each other. Each of the at least two second image slices can correspond to an individual image slice of the first image pair 102. For example, the coronal image slice 120 of the second image pair 118 can correspond with the coronal image slice 106 of the first image pair 102 to establish a first image set 103, including the coronal image slices 106 and 120. Also, the sagittal image slice 122 of the second pair 118 can correspond with the sagittal image slice 108 of the first image pair 102 to establish a second image set 105, including the sagittal image slices 108 and 122. Thus, while depicted as first and second image pairs 102 and 118, the adaptive image-guided therapy system can handle the images slices as a coronal, first image set 103 and a sagittal, second image set 105 without establishing, grouping, or otherwise recognizing any correspondence between coronal/sagittal “pairs”, such as first image pair 102 and second image pair 118. Thus, first image pair 102 and second image pair 118 can be provided merely for illustration and need not be established by the adaptive image-guided therapy system. A first initial trajectory, such as a first motion vector 124, can be identified based on an interpolation of respective registration points of the specified anatomical structure 104 for coronal slice 120 and the coronal image slice 106, e.g., the first image set 103. A second initial trajectory can similarly be identified, such as a second motion vector 126, based on an interpolation of respective registration points of the specified anatomical structure 104 for the sagittal image slice 122 and the sagittal image slice 108, e.g., the second image set 105. Here, the second initial trajectory can also be in the in-plane direction. A third initial trajectory, such as a third motion vector 128, can be determined based on the first and second initial trajectories. Here, the third initial trajectory can be determined in a through-plane (e.g., the third plane 116) direction oriented as substantially orthogonal to both of the two respective planes (e.g., the first plane 112 and the second plane 114).

The process described above with respect to time points A and B can be subsequently repeated, such as with respect to time points B and N. For example, the adaptive image-guided therapy delivery system can receive, during the radiation session, a third image “pair” and similarly regress the third image pair against the second image pair 118, as described above where the second image pair 118 was regressed against the first image pair 102. Also, the regression of the third image pair against the second image pair 118 can be similarly used to determine a trajectory in the through-plane direction at or near time point N. Similar to that described above with first and second image pairs 102 and 118, the third image pair need not be actually grouped or established with respect to each other and can be treated by the adaptive image-guided therapy system independently in respective coronal and sagittal regressions. As indicated by the ellipses, a similar process can be repeated multiple times with respect to a time point N−1 (e.g., time point B) and time point N. Alternatively or additionally, at 134, subsequent estimations of motion in the through-plane direction can be used to establish or adjust the third initial trajectory.

In an example, a machine learning model 130 can be trained using training data obtained during the radiation session. For example, the training data can include the first, second, and third trajectories, such as the first motion vector 124, the second motion vector 126, and the third motion vector 128. Here, the model can be used such as to estimate a 3D position (including a through-plane coordinate) of the specified anatomical structure 104 at a present time point (e.g., time point N). the model can also be used such as to predict movement of the specified anatomical structure 104 at or near a future time point N′. The model can also be used to estimate past movement, such as establishing or adjusting the third motion vector 128, in the through-plane direction. For example, movement of the specified anatomical structure 104 can be established or adjusted using the trained model, the movement relative to at least one radiation aperture of the adaptive image-guided therapy delivery system.

In an example, the adaptive image-guided therapy delivery system can compare predicted movement, such as movement predicted by using the model at or near a future time point (e.g., to predict movement at time point N′ at time point A, time point B, or time point N), with actual motion subsequently determined at time point N′ via interpolation. Such a comparison can yield a prediction error, and the prediction error can also be used as an input to the machine learning model. Such feedback can help retrain the model as well as enhance performance of the model.

A benefit to the above-described approach is that, using the model, movement can be determined in the first plane slice 112, the second plane slice 114, and the third plane slice 116 at a relatively equal rate, which in turn can help mitigate undesired abrupt transitions between frames during motion tracking. For example, the movement can be determined based on the model, in an in-plane direction at a latency equal to a latency for movement determined in the through-plane direction. This can allow for controlling of delivery of a radiation therapy beam at approximately symmetrical margins for the in-plane direction and the through-plane direction, such as improving a continuity and smoothness of the delivered radiation.

As depicted in FIG. 1B, once the machine learning model 132 is trained, the adaptive machine image-guided therapy delivery system can infer the through-plane activity (or 3D position including a through-plane coordinate) using a sequence of image slices (e.g., the first image set 103) taken from a single plane and without using images from any second plane at an angle to the first plane. Here, the model can be used to infer the through-plane activity, such as motion of the specified anatomical structure 104, at a time point N′, where the time point N′ is after the time point N. For example, the model can be used to infer motion of the specified anatomical structure 104 at a time point N′, where the time point N′ is after the time point N by a latency period (e.g., one to three seconds) sufficient to allow for repositioning the radiation aperture if necessary. Here, the model can be used to infer motion of the specified anatomical structure 104 at the time point N′, such as in the through-plane direction and/or in the in-plane direction of a second plane at an angle to the first plane, without receiving any through-plane or second plane cine image slices at the time point N′.

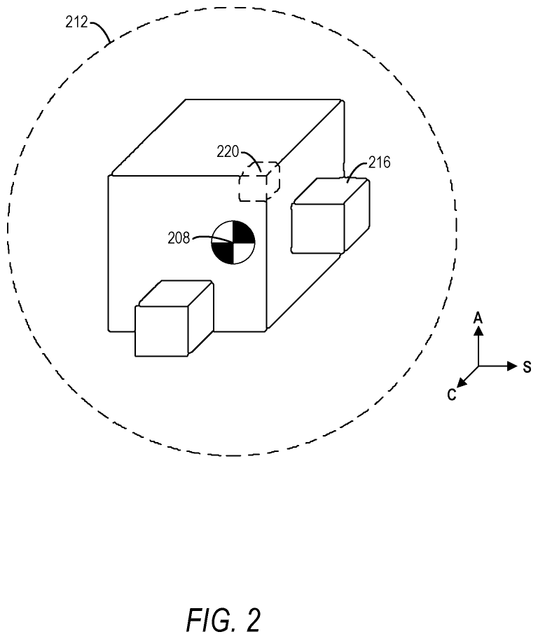

FIG. 2 illustrates generally a view of an object or target locus 216, such as a tumor region to be treated using radiation therapy. Information indicative of the target locus 216 can be obtained using a volumetric imaging technique. The representation in FIG. 2 is shown has having rectangular facets merely for purposes of illustration, and an actual target locus 216 can include curved or irregular surfaces. A region of interest 212 can be imaged, such as using one or more of MR imaging, CT imaging, pseudo-CT image visualization, portal imaging, ultrasound imaging, or other techniques. The target locus 216 can be identified such as using automated, manual, or semi-automated image segmentation techniques. For example, a specified label can be assigned to voxels (e.g., a voxel 220) to identify a locus or group of voxels comprising a radiation therapy target. As an illustrative example, a contrast value for a voxel 220 can be compared to adjacent voxels, and an edge of a tumor or other region can be identified such as using contrast information. Other regions can also be identified such as nearby organs or locations of other features.

Various features of a target locus can be determined such as one or more edge locations (e.g., using an edge detection or edge identification technique). For example, a position of the target locus 216 can be identified by determining a spatial centroid 208 of the target locus 216. The use of a centroid 208 as an extracted feature is illustrative and other features can be used for tracking target locus 216 motion, such as a manually identified or automatically determined location of a point, surface, or edge of the target locus 216. In yet another example, an implantable or external seed fiducial can be used, such as providing an indicium in obtained imaging information.

The target locus 216 and one or more corresponding features such as the centroid 208 can be identified during treatment planning imaging, such as inter-fractionally (e.g., between radiation therapy delivery sessions) or just prior to beginning a radiation therapy delivery session. Volumetric imaging information comprising the target locus 216 can be referred to as a “reference image.” As mentioned above, a challenge can exist because image acquisition and processing latency can preclude “real time” acquisition of volumetric imaging information during radiation treatment. Accordingly, intra-fractional image acquisition (such as during or between radiation therapy deliveries in a radiation therapy session) can include more rapid acquisition of imaging slices (including one or more of one-dimensional profiles, two-dimensional slices, cine images, or three-dimensional volumes comprising a sub-volume or sub-region of an earlier-imaged volumetric region). Information indicative of a portion of the target locus obtained rapidly from one or more imaging slices can be compared to information obtained from an earlier-acquired volumetric reference image to update a therapy protocol, such as to adjust therapy due to movement of the target locus. The imaging modality used for obtaining or generating the volumetric reference image need not be the same as is used for intra-fractional imaging.

In another example, the target locus 216 need not be segmented or otherwise identified in the volumetric reference image. Use of the phrase “target locus” is merely illustrative of a target within the region of interest such as a portion or an entirety of a tumor to be treated or other anatomical structures such as organs near the tumor. In addition to, or instead of segmentation of the target locus 216, various techniques as described herein can be performed such as including spatially registering a portion of the volumetric reference image (e.g., a specified region of interest) or an entirety of the volumetric reference image with other imaging information such as using voxel values including contrast or grayscale values. For example, such spatial registration can include three-dimensional (e.g., volumetric) registration with one or more two-dimensional imaging slices (e.g., 3D-to-2D registration), or other techniques.

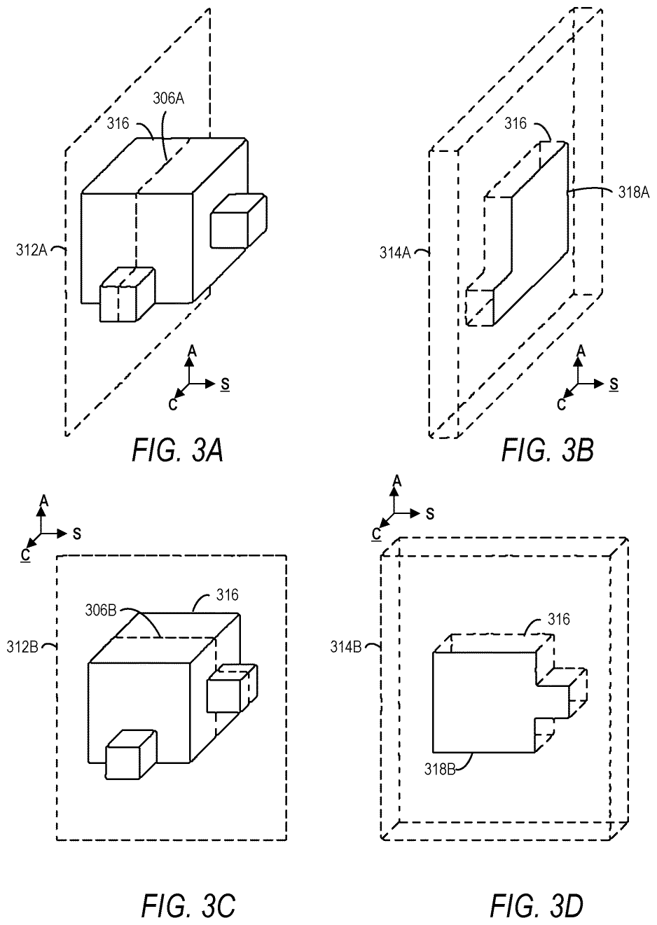

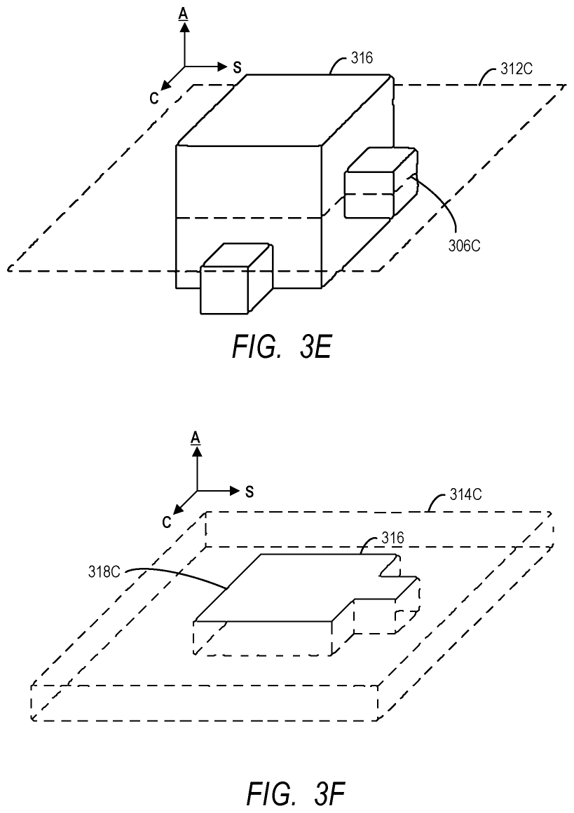

FIG. 3A and FIG. 3B illustrate views of an imaging acquisition plane 312A (in FIG. 3A) of a target locus 316 and a corresponding acquired imaging slice 314A (in FIG. 3B) corresponding to a first imaging plane orientation intersecting the target locus 316 along a line 306A. As an illustrative example, FIG. 3A can represent a sagittal orientation of the image acquisition plane 312A and corresponding slice 314A. An imaging “slice” can include two-dimensional pixel imaging information or three-dimensional imaging information, such as having a small finite thickness as shown illustratively in FIG. 3B. A portion 318A of the target locus in the imaging slice 314A can be identified, such as again using a segmentation technique, for example using a discrete dynamic contour, snake, or level set, or a registration-based technique.

A series of imaging slices can be obtained, such as including different imaging acquisition plane orientations as shown in FIG. 3C, FIG. 3D, FIG. 3E, and FIG. 3F. For example, FIG. 3C and FIG. 3D illustrate views of an imaging acquisition plane 312B (in FIG. 3C) intersecting the target locus 316 along a line 306B and a corresponding acquired imaging slice 314B (in FIG. 3D) including a different portion 318B of the target locus 316, corresponding to a second imaging plane orientation, such as can be orthogonal to the first imaging plane orientation mentioned above in relation to FIG. 3A and FIG. 3B. As an illustration, FIG. 3C and FIG. 3D can correspond to a coronal plane. FIG. 3E and FIG. 3F illustrate views of an imaging acquisition plane 312C (in FIG. 3E) intersecting the target locus 316 along a line 306C and a corresponding acquired imaging slice 314C (in FIG. 3F) including yet another different portion 318C of the target locus 316, corresponding to a third imaging plane orientation, such as can be orthogonal to one or more of the first and second imaging plane orientations mentioned above in relation to FIG. 3A, FIG. 3B, FIG. 3C, and FIG. 3D. As an illustration, FIG. 3E and FIG. 3F can correspond to an axial imaging plane. The imaging slices of FIG. 3A, FIG. 3B, FIG. 3C, FIG. 3D, FIG. 3E, and FIG. 3F are shown as two-dimensional planes that are orthogonal to each other. However, as mentioned in relation to other examples herein, the slices need not be planar nor orthogonal. Each acquired imaging slice can be processed, including processing of one or more slices in parallel with an acquisition of an imaging slice. For example, such processing can include sampled information from a volumetric reference image, such as corresponding to a plane or region of the acquired imaging slice.

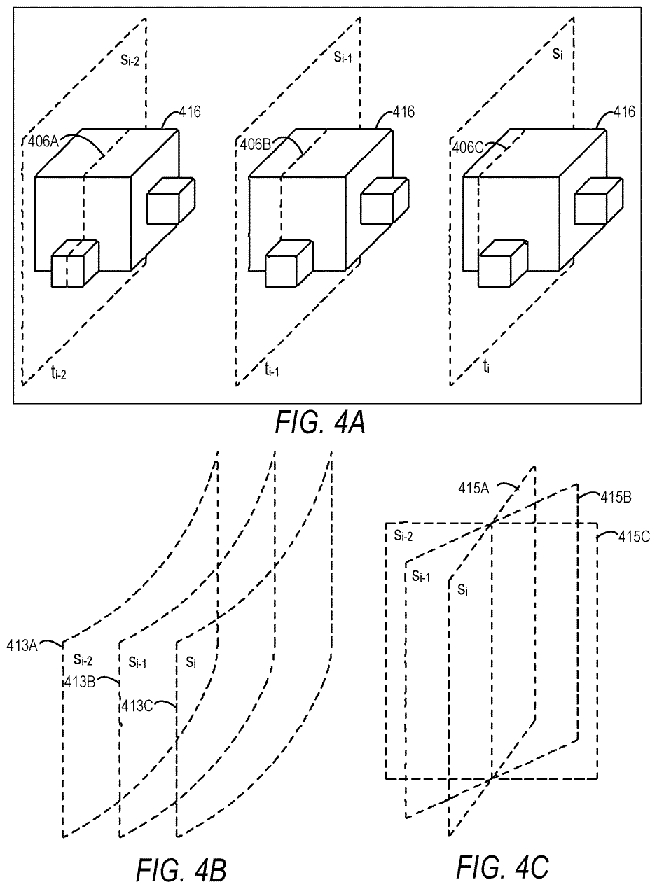

In one approach, a feature extracted from one or more of the imaging slices 314A, 314B, or 314C can be compared with imaging information extracted from a volumetric reference image. For example, a segmented portion of the target locus 316 from the volumetric reference image, or a region encapsulating the target locus and surrounding tissues, can be spatially registered with a corresponding imaging slice 314A, 314B, or 314C to determine if the target locus has shifted. However, comparing a feature extracted from a single two-dimensional imaging slice with a corresponding feature from the volumetric reference image can present challenges. For example, FIG. 4A illustrates generally a series of imaging acquisition planes 412A, 412B, and 412C, such as can be obtained as a target locus 416 moves from one imaging acquisition instance to another. At time ti−2, a line 406A where the imaging acquisition plane intersects the target locus 416 is roughly centered, and at time ti−1, the line 406B is shifted, and at time ti, the line 406C is shifted further. Imaging slices obtained at times ti, ti−1, ti−2, . . . can be labeled as Si, Si−1, Si−2, . . . generally.

If the motion of the target locus is out-of-plane, the centroid or other feature location cannot appear to shift significantly from image-to-image, even though the target locus has moved significantly, or the target locus can appear to deform, making interpretation of motion difficult. However, such out-of-plane motion can be properly tracked such as by one or more of varying an orientation of successively acquired imaging slices (e.g., as shown in FIG. 4B and elsewhere), or varying or compensating for a non-planar shape of the imaging acquisition region so that the imaging slices are not represented as perfectly planar in a three-dimensional sense (e.g., as shown in FIG. 4B). For example, FIG. 4B illustrates generally a series of imaging slices 413A, 413B, and 413C, such as can include a curved shape. Generally, even when referring to image acquisition “planes,” the techniques described herein can be used with other slice geometries such as the curved slices of FIG. 4B. As an illustrative example, MR imaging information can be available as planar slices, and a transformation can be applied to the planar slices to obtain surfaces that curve in a three-dimensional sense, such as to help capture or correct for distortion across the imaging field such as to compensate for inhomogeneity in an established magnetic field. As another illustrative example, FIG. 4C illustrates generally a series of imaging acquisition planes 415A, 415B, and 415C such as can include different orientations that need not each be orthogonal.

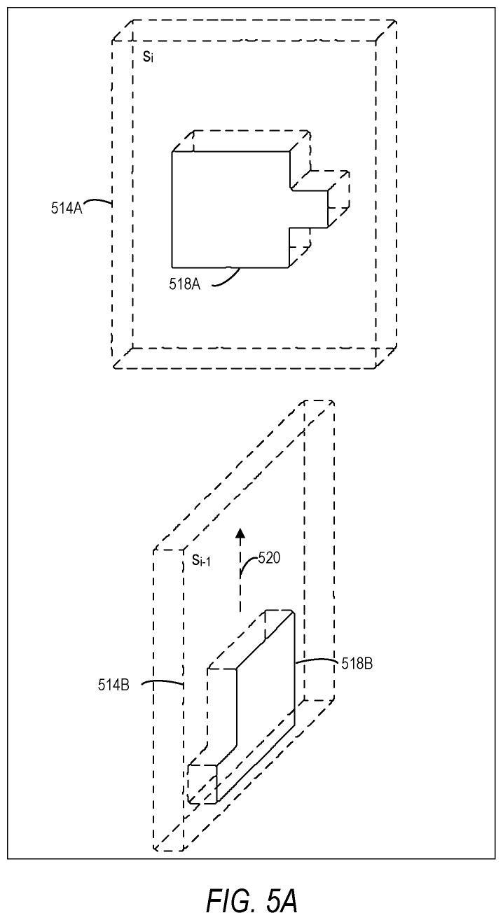

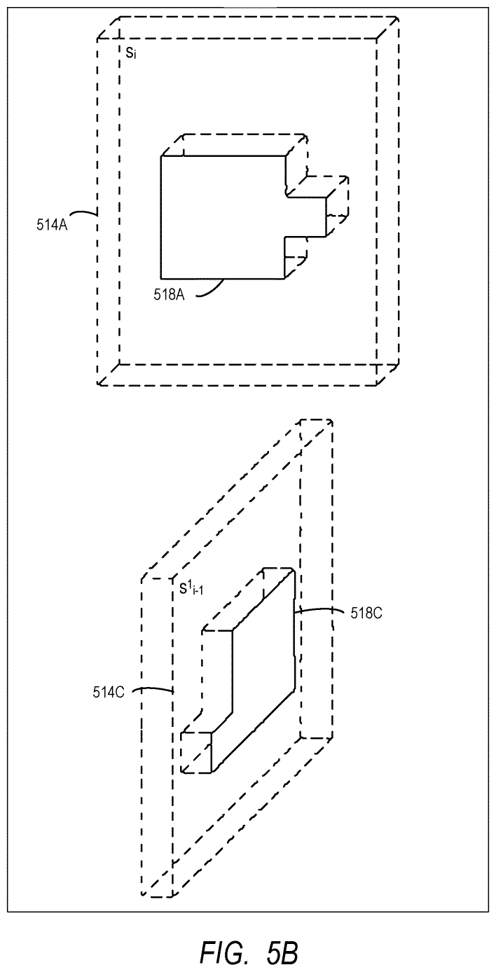

As mentioned above, if imaging slices are acquired sequentially in time, the target locus is likely to shift between successive image acquisitions. FIG. 5A illustrates generally a series of two imaging slices, such as including a target locus that is displaced between first and second imaging slices. The example of two sequentially acquired imaging slices is illustrative. The techniques described herein are also generally applicable to sequences of more than two imaging slices (or even a single imaging slice analyzed with respect to a portion or an entirety of the reference volumetric imaging information).

An imaging slice 514A (e.g., Si) can include a portion 518A of the target locus, and a second imaging slice 514B (Si−1, such as acquired earlier in time) can include a second portion 518B. The second portion 518B appears displaced with respect to the first portion 518A by a displacement 520. Because the displacement 520 is generally an unknown value or vector, various techniques can be used such as to spatially register the first and second portions 518A and 518B. As shown in FIG. 5B, a spatially registered slice 514C can include an adjusted location of a portion 518C of the target locus. Notationally, a set or series of acquired imaging slices can be labeled Si, Si−1, Si−2, . . . , Si−n, with Si corresponding to the most recently acquired slice for processing and Si−n corresponding to the earliest-acquired slice for processing. A series of spatially registered imaging slices can be labeled Si, S′i−1, S′i−2, . . . S′i−n.

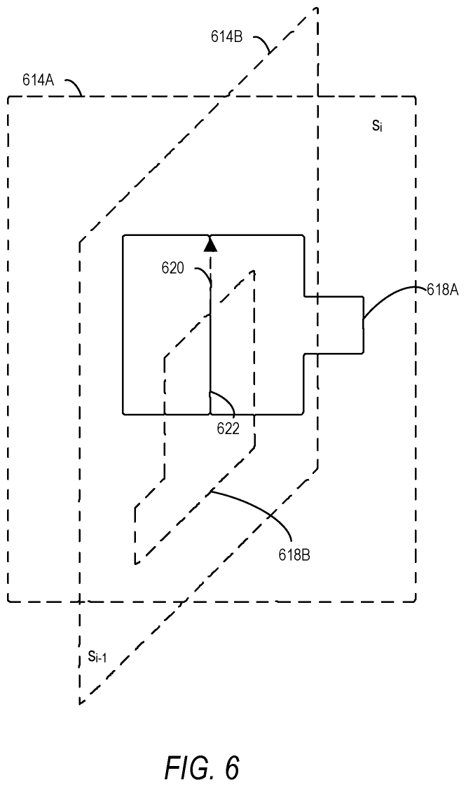

FIG. 6 illustrates an example of segmented portions of a target locus from two (or more) imaging slices Si, Si−1, Si−2, . . . , Si−n. In one approach, a metric can be defined that quantifies how well the two portions 618A and 618B of a target locus are aligned (or more generally, how well two imaging slices 614A and 614B or imaging volumes are aligned), such as the similarity between the pixels along the intersection line 620 joining the first portion 618A and the shifted second portion 618B. Examples of such metrics include one-dimensional normalized cross-correlation or mutual information. An automated optimization technique can be executed to find translations of the second portion 618B that provide the optimal value of the metric. A mask can be used to limit the calculation to the most relevant pixels only, such as pixels close to the target of interest. This first approach generally relies on the planes having a common intersection line.

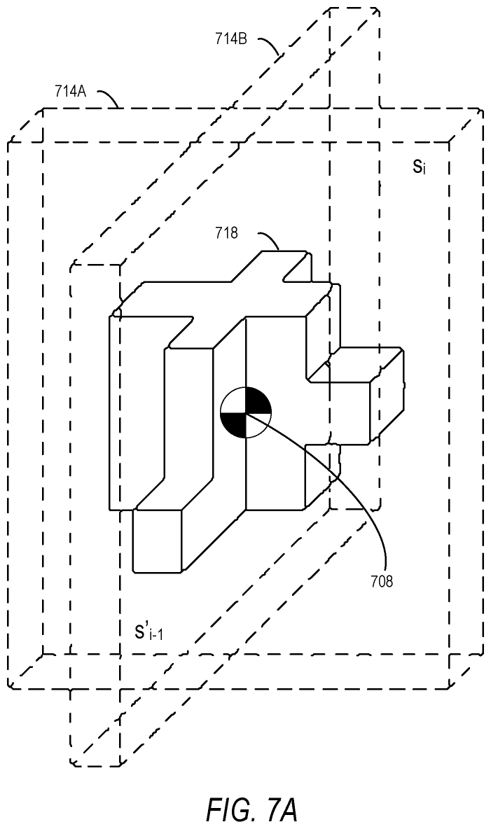

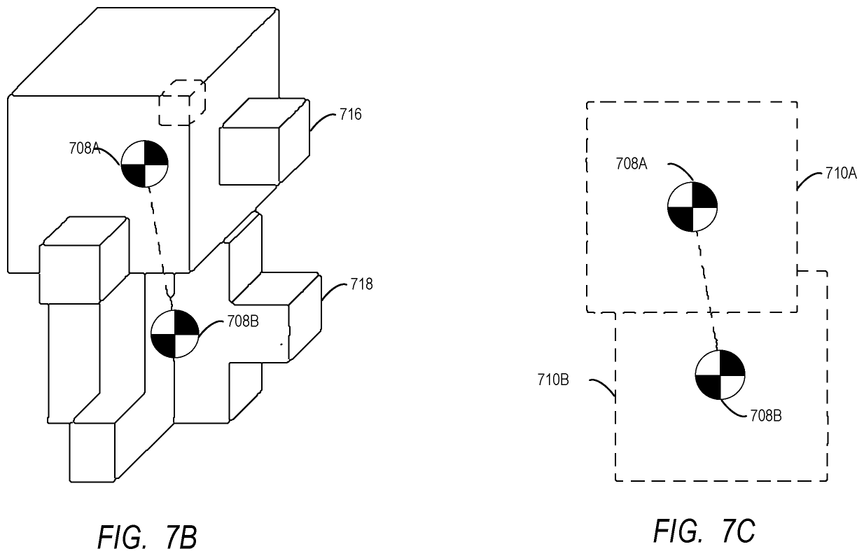

Another approach can include creating three-dimensional (3D) voxel images, such as having a first image corresponding to a first slice orientation including populated voxels corresponding to a portion of the target locus within the slice and zero-valued voxels outside the slice, and a second image of a second slice orientation including its own populated voxels corresponding to the portion of the target locus in the second image. As an illustrative example, MR imaging slices generally have a finite thickness, such as corresponding to about 5 millimeters. A finite slice thickness can be taken into account by assigning voxels with slice information within the actual slice thickness using two-dimensional (2D) pixel information extracted from an MR imaging slice. A 3D registration technique can be used find the optimal translation between the two 3D images, and masks can be used to exclude voxels that do not contain slice information. The 3D registration approach also generally relies on having a common intersection line. An illustrative example of three-dimensionally-registered imaging slices is shown illustratively in FIG. 7A.

In yet another approach, a prediction technique can be applied to a prior imaging slice to find the most likely position of a portion of the target locus for the prior imaging slice orientation but having the same time stamp as the current or a more-recently-acquired imaging slice. Various techniques can be used for such prediction, such as Kernel Density Estimation, a neural network, a deep learning neural network model, regression technique comprising a support vector machine or random forest, or a template matching technique. Prediction can be particularly effective if a motion of the target locus is highly periodic in nature, such as associated with respiratory motion. Although using a prediction is an approximation only, it can also be used in combination with the image-based methods described above and elsewhere herein. A prediction-based approach does not rely on image acquisition planes having a common intersection line, and so prediction techniques can be used when parallel slices are acquired.

In yet another approach, when fast perpendicular MR-imaging slices are acquired, dark lines can appear in the current image in the location where the previous image was excited and not yet fully relaxed to equilibrium. Because the excited magnetic dipoles from the previous slice follow the moving anatomy, a visible previously excited region (e.g., an excitation line) can be used as an anchor to align previous slices with later-acquired imaging information. This can be taken into account implicitly with the other techniques mentioned above, because such darkened regions as pixel or voxel features can affect registration, or explicitly, such as by identifying or explicitly determining the location of such darkened regions in acquired imaging slices. For example, a peak-detection technique can be used to find points along the center of the darkened regions, and a line (straight or curved) can be found that provides a best fit to these points. Various examples described in this document refer to registration between images, such as including registration of a segmented portion of a first image with a segmented portion or other identified features of a second image (or a series of images). Generally, a registration between two images can include a moving and a fixed image. The goal can be to determine a position or transformation of the moving image such that it best fits the fixed image. “Move” can mean, for example, to shift the moving image spatially in one or more dimensions (translations only), shift and rotate the moving image, or even deform the moving image. The degrees of freedom available to move the moving image can be referred to as “optimization parameters,” which, as an illustrative example, can include two degrees of freedom in the case of shift only with two 2D images, or 6 degrees of freedom for shifts plus rotations for 3D images. Even though we can be generally referring to 3D image registration, “optimization parameters” can still be generally referred to as “shift values” without loss of generality.

For a given set of optimization parameters, a metric can define a fit of a determined overlap between the moving and fixed images. Various examples can include determining a normalized cross-correlation or mutual information (such as using voxel or pixel values as input values), and such techniques can provide an indication of an optimal value of the metric when the images match perfectly. The nature of the underlying imaging technique can be helpful in establishing which metric to use. For example, a mutual information technique can be used for images obtained using different modalities such as positron emission tomography (PET) and CT imaging.

An optimization technique can be used to find the shift values that optimize the metric between the moving and fixed images, such as to identify shift values that give the best match between the two images. Different optimizers that can be used can include gradient descent, simulating annealing, trust-region gradient descent, or one or more other techniques. In some cases, there can be deformations between the images, but it is not always necessary to optimize deformation vector fields and take these into account, because optimization of deformation information can result in one or more of undesired latency or can erode stability of the optimization technique. Calculation of the metric can be limited to a specified region of interest, such as referred to as a “mask.” For example, assuming there is negligible deformation in the region of interest, one or more of translations or rotations, without deformation, can be used to determine shift values that are locally optimal in the specified region of interest.

Generally, if one or more 2D imaging slices are available, a motion of a radiation therapy target can be determined between a current 2D slice (such as an MR imaging slice) and an initial 3D volume (e.g., a reference volumetric image). As an illustrative example, the 3D volume can also correspond to a particular phase of respiration to reduce respiration blurring, which can be accomplished either with gated or triggered imaging (e.g., triggered imaging), or by extracting the 3D volumetric image from four-dimensional imaging information at a particular phase (e.g., extracting a 3D volumetric snapshot from a 4D imaging series, such as 4D imaging information obtained using MR imaging).

In one approach, the target itself can be identified in an imaging slice. However, while segmentation is mentioned elsewhere herein, the techniques described herein are applicable when segmentation or other feature extraction is not performed. As an illustrative example, a best fit of grayscale values can be determined between one or more later-acquired imaging slices and an earlier-acquired reference 3D image. A target does not need to actually be in the image, but a region of interest can be specified, such as can include a portion of the target, or the target plus a margin, or just an arbitrary region of interest without the target.

In one approach, a 2D/3D registration can be performed to find a relative shift between a current imaging slice and the reference volume. The 3D image can represent the ‘moving image’ and a 2D slice can represent the ‘fixed image’ in relation to the registration technique. Generally, the location of the target can be identified in the 3D image, and applying the relative shift to the earlier target location can provide an estimate of an updated location of the target, such as within a three-dimensional coordinate frame. Such an estimate can be performed without requiring determination of deformation, and can even be performed without use of rotational parameters, such as to increase execution efficiency of the optimization technique. Such simplifying assumptions can be applied such as when registering a 3D reference image to one or more 2D imaging slices.

A specified region of interest can be used to define a mask around the target plus a margin, for example (though the region of interest need not include the target or an entirety of the target). Deformation can be present, but as an illustrative example, if the margin is small, deformation will also be small (e.g., negligible) and a shift of the target itself can be determined using a registration technique ignoring deformation. In this manner, a single plane technique (e.g., a technique where the imaging slice is the fixed image) can still provide information indicative of a shift in three dimensions. Such a technique is robust when there are enough features in the images to “lock in” the registration in three dimensions—the registration converges to a well-defined optimum—though the features need not be extracted or segmented.

In one approach, a normalized cross-correlation of the gradient of the images can be used as a registration metric, since such a technique tends to match image edges and is less sensitive to absolute grayscale values that can differ from image to image. A single imaging slice or plane can, in some cases, not be sufficiently robust to establish a shift with a clear optimal value (colloquially, ‘lock in’). Accordingly, additional slices can be useful to help lock in all directions, for example using a current sagittal and a previously acquired coronal slice, or vice-versa, as illustrative examples. As a count of slices used for registration increases, whether parallel, orthogonal, or in non-parallel and non-orthogonal planes, more information is available to perform a registration between the reference volumetric image and the later-acquired imaging slices, which improves the robustness of the resulting shift parameter determination.

FIG. 7A illustrates generally a composite 718 representation of a target locus, such as after spatial registration of segmented portions of the target locus acquired at different times corresponding to spatially registered imaging slices 714A and 714B. After portions of the target locus from two or more imaging slices have been spatially registered, a feature can be extracted such as from a composite 718. For example, a location of a centroid 708 of the composite 718 or other feature can be determined, such as providing an estimate of a centroid location of the target locus for use in updating a radiation therapy protocol. In another example, a determined spatial displacement between portions of the target locus in the first and second imaging slices 714A and 714B can be used to update a radiation therapy protocol.

FIG. 7B is a depiction showing a difference between a later estimated or predicted location of a composite 718 of an example of a target locus. For example, such a difference can be estimated or predicted according to acquired spatially registered imaging information as compared to an earlier location of the target locus 716 such as represented by earlier-acquired volumetric imaging information. A displacement of one or more features can be used to estimate a present or future target locus location. For example, a displacement can be determined between an earlier-determined centroid 708A extracted from the reference imaging information and later-determined centroid 708B. In another example, shift parameters from an imaging registration can be used to provide an updated target locus without requiring extraction or determination of features such as a centroid. Generally, a variety of other techniques can be used such as to extract information indicative of a motion or displacement of the target locus 716 using spatially registered imaging slices.

For example, once a set of previous imaging slices are aligned to the current slice to form a slice set Si, S′i−1, S′i−2, . . . S′i−n, an optimal registration between the slice set (or a composite 718 generated from such a slice set) and a three-dimensional reference volume can be found (such as a reference volume corresponding to the target locus 716 as shown in FIG. 7B). As an illustrative example, a sagittal slice acquisition can be preceded by a coronal slice acquisition. A spatial translation can be identified that brings the coronal slice to the same time point as the sagittal slice to spatially register the coronal slice with the sagittal slice. Then, both slices can be established within a 3D voxel coordinate space. This can be done by inserting the slices as infinitesimally thin slices, or as finite slices using the known slice thickness (such as shown in the composite 718 of FIG. 7A and FIG. 7B).

Voxels that are not filled by portions of the target locus from each slice can be left unfilled. A 3D-to-3D registration can then be determined between the 3D reference image (e.g., corresponding to target locus 716) and the 3D “slice-filled image” (corresponding to the composite 718). A mask can be used to filter out voxels that have not been filled. In another example, a 3D-to-2D registration can be performed, such as using multiple 2D slices rather than a single composite. For example, shift parameters (e.g., displacement of one or more features such as a centroid or a set of shift values specifying a translation or rotation of region of interest) of the 3D reference image can be evaluated, such as optimizing values of a similarity metric that compares the voxels of the shifted 3D volumetric reference image to each of the slices of the multiple 2D imaging slices, using registration and optimization techniques as mentioned above. In this manner, a 3D-to-2D registration can be performed, such as to identify an optimal set of shift values. A location of the target can be updated using information obtained from the shift values. 3D-to-3D, 2D-to-2D, or 3D-to-2D registration techniques need not require image segmentation (e.g., the target locus itself need not be segmented), and such registration need not require identification of imaging features such as a centroid or edge location. Instead, registration can be performed using grayscale or contrast imaging information (or other extracted values), such as over a specified region of interest generally as mentioned elsewhere herein.

FIG. 7C is a depiction showing a shift of an example of a therapy locus to a new location. An earlier radiation therapy target region 710A can be established, such as corresponding to a target locus extracted from a volumetric reference image or established according to other treatment or dosimetric objectives. Such a therapy target region 710A can be established at least in part using one or more of positioning a radiation therapy output, modulating a therapy beam including modulating one or more of intensity or beam shape, or moving the patient using an actuator such as a moveable therapy couch or platform. One or more features can be extracted from the therapy target region, such as a centroid 708A. An estimated or predicted displacement of the target locus (e.g., a tumor or other structure to be treated) can be determined, such as using other techniques as described herein.

The therapy target region 710A can be adjusted to provide an updated therapy target region 710B. For example, if the target locus has been translated due to patient motion, a similar displacement can be applied to the earlier region 710A to provide an updated therapy target region 710B. Similarly, if other features are extracted, the therapy target region 710A can be adjusted using other techniques such as rotation or scaling. In another example, instead of or in addition to adjusting the therapy target region 710A, other techniques can be used to control therapy, such as gating therapy to inhibit delivery of radiation therapy unless an updated therapy target region 710B falls within a specified zone. In yet another example, a surrogate signal, such as derived from a sensor output, can be used to gate therapy. For example, a sensor output can be correlated with the location of a feature extracted from imaging slices, such as a centroid location of a target locus within the imaging slices after the imaging slices are spatially registered. Therapy delivery can be gated in a synchronous manner, such as triggered in response to the sensor output (e.g., such as to deliver therapy at a certain time during a periodic motion such as respiration).

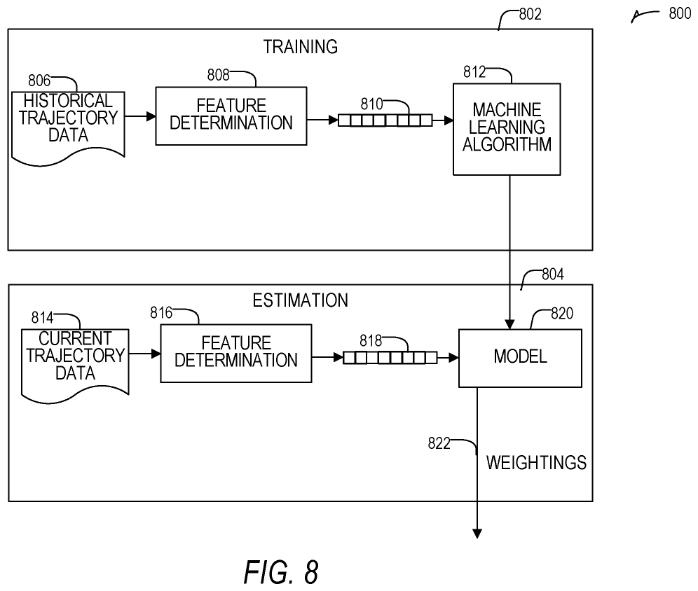

FIG. 8 illustrates an exemplary regression model machine learning engine 800 for use in estimating motion in a through-plane direction, predicting 3D motion, or both. Machine learning engine 800 utilizes a training engine 802 and an estimation engine 804. Training engine 802 inputs historical trajectory data 806 (e.g., past interpolations from in-plane cine images) into feature determination engine 808.

Feature determination engine 808 determines one or more features 810 from this historical trajectory data 806. Stated generally, features 810 are a set of the information input and include information determined to be predictive of a particular outcome. The features 810 may be determined by hidden layers, in an example. The machine learning algorithm 812 produces a predicted motion model 820 based upon the features 810.

In the estimation engine 804, current trajectory information 814 (e.g., presently interpolated from in-plane cine images) may be input to the feature determination engine 816. Feature determination engine 816 may determine features of the current information 814 to estimate a through-plane trajectory or a predicted future trajectory. In some examples, feature determination engines 816 and 808 are the same engine. Feature determination engine 816 produces feature vector 818, which is input into the model 820 to generate one or more criteria weightings 822. The training engine 802 may operate in an online manner to train the model 820. It should be noted that the model 820 may be periodically updated via additional training or user feedback (e.g., additional, changed, or removed trajectories).

The machine learning algorithm 812 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 802.

In an example, a regression model is used and the model 820 is a vector of coefficients corresponding to a learned importance for each of the features in the vector of features 810, 818. In an example, the machine learning algorithm 812 implements a regression problem (e.g., linear, polynomial, regression trees, kernel density estimation, support vector regression, random forests implementations, or the like).

In some instances, an error calculated by comparing predicted motion, based on the model 820, with actual subsequent motion may be used to update or retrain the model 820. In some instances, a calculation may be performed to determine whether the current trajectory information 814 is consistent with corresponding predicted trajectories previously predicted by the model 820, and pause the treatment if it is not. When the treatment is paused, a new model 820 may be generated, or the old model 820 may be reused if the measurement (e.g., motion) was an aberration.

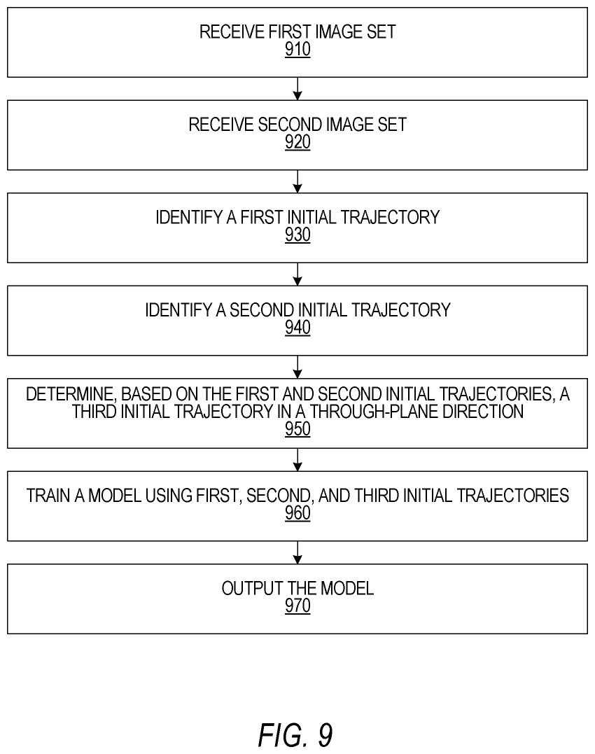

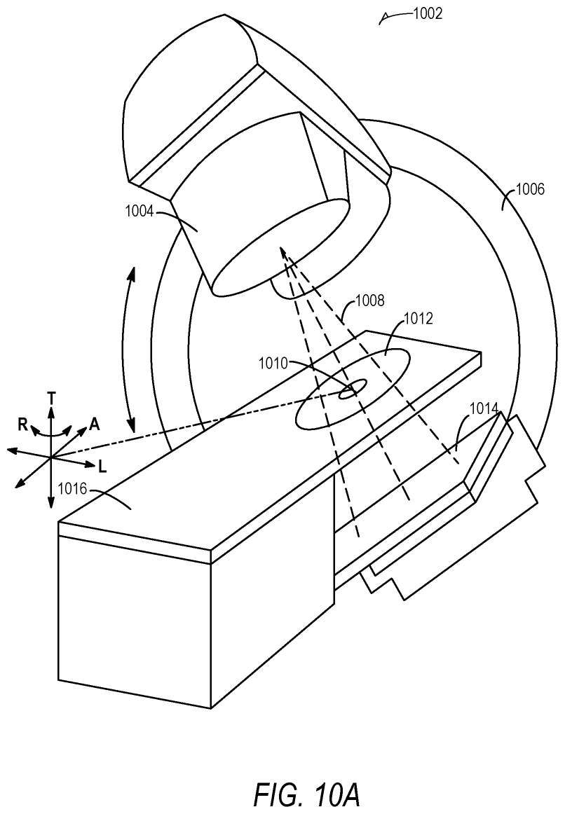

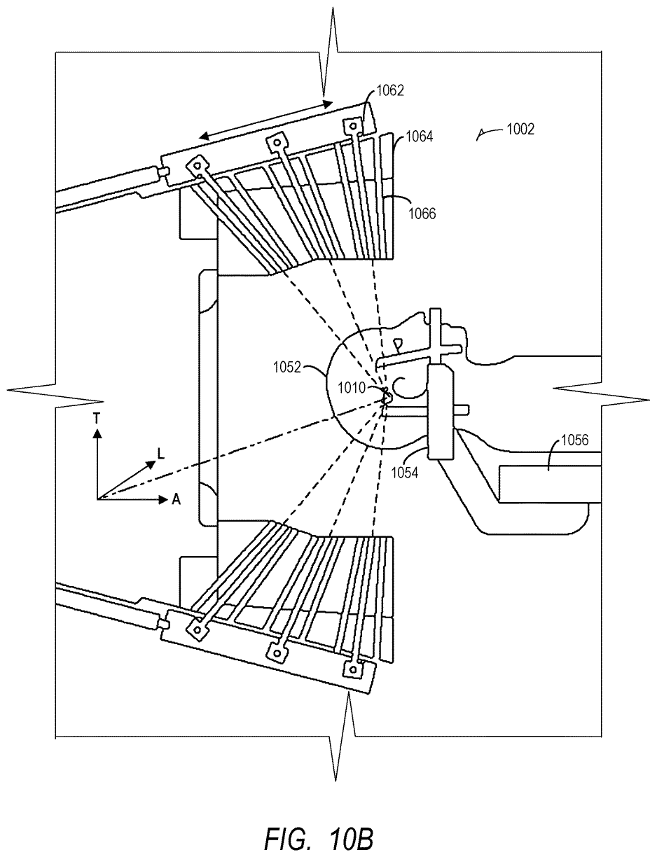

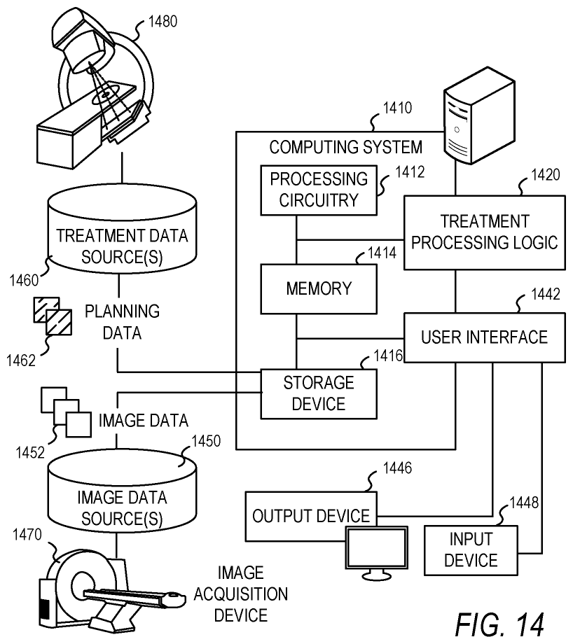

FIG. 9 is a flowchart that describes a method of using an adaptive image-guided therapy delivery system. The operations discussed in FIG. 9 can be performed in sequence, in parallel, skipped, or out of order. The operations discussed in FIG. 9 can be performed by computing system 910 (FIG. 14). In an example, at 910, the method can include receiving, during a radiation session, a first image set corresponding to a specified anatomical structure of the patient, the first image set of the specified anatomical structure captured at different times at a first plane.

At 920, the method can include receiving, during the radiation session, a second image set corresponding to the specified anatomical structure of the patient, the second image set of the specified anatomical structure captured at different times at a second plane oriented at an angle to the first plane.

In an example, at 930, the method can include identifying a first initial trajectory based on an interpolation of respective registration points of the specified anatomical structure for first and second image slices of the first image set, the first initial trajectory having an in-plane direction with at least one of the first and second planes. For example, an interpolation can be calculated between at least one initialization image and a template, the at least one initialization point obtained during a preparation session performed before the radiation session. Predicting the first and second trajectories can be based on the interpolation between the at least one initialization point and the template.

At 940, the method can include identifying a second initial trajectory based on an interpolation of respective registration points of the specified anatomical structure for first and second image slices of the second image set, the second initial trajectory in an in-plane direction with at least one of the first and second planes.

In an example, at 950, the method can include determining, based on the first and second initial trajectories, a third initial trajectory in a through-plane direction oriented substantially orthogonal to both of the first and second planes.

At 960, the method can include training a model, using training data obtained during the radiation session, the training data including the first, second, and third trajectories, to estimate current or real-time 3D position (including a through-plane coordinate) of the specified anatomical structure. Training the model can include regressing the first trajectory, the second trajectory, and the third trajectory. Also, determining movement of the specified anatomical structure can include offsetting movement of the aperture relative to the specified anatomical structure, the offsetting identified using the trained model. The method can also include determining an error between one of the first or second trajectories and a predicted trajectory output by the model. For example, the model can be retrained based on feedback of the determined error. Movement can be determined, based on the model, in an in-plane direction at a latency equal to a latency for movement determined in the through-plane direction. Also, delivery of a therapy beam can be controlled at approximately symmetrical margins for the in-plane direction and the through-plane direction.

At 970, the method can include outputting the model for use in generating an updated therapy protocol. For example, an updated therapy protocol can be generated such as to control delivery of a beam based on the movement.

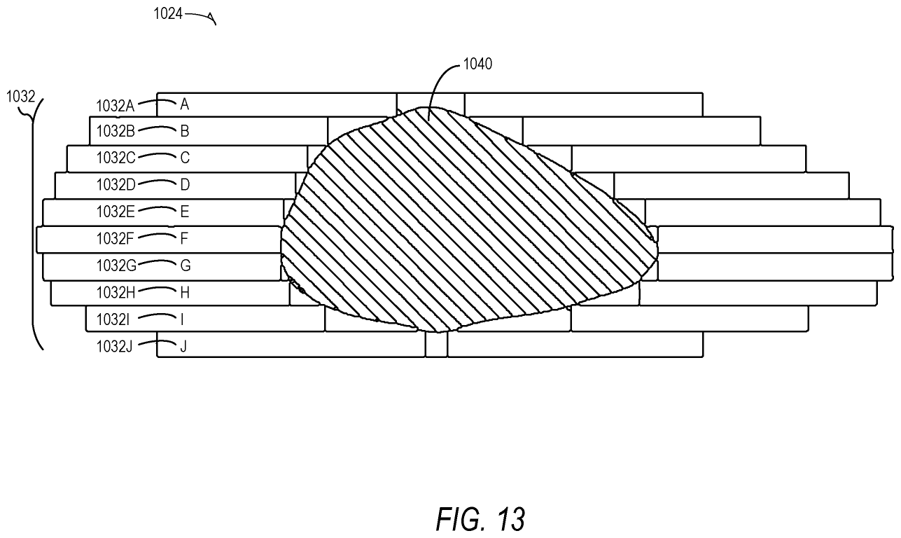

FIG. 10A illustrates generally an example of a radiation therapy system 1002 that can include radiation therapy output 1004 included such as to provide a therapy beam 1008. The radiation therapy output 1004 can include one or more attenuators or collimators, such as a multi-leaf collimator (MLC) as described in the illustrative example of FIG. 13. Referring back to FIG. 10A, a patient can be positioned in a region 1012, such as on a platform 1016 (e.g., a table or a couch), to receive a radiation therapy dose according to a radiation therapy treatment plan. The radiation therapy output 1004 can be located on a gantry 1006 or other mechanical support, such as to rotate the therapy output 1004 around an axis (“A”). One or more of the platforms 1016 or the radiation therapy output 1004 can be moveable to other locations, such as moveable in transverse direction (“T”) or a lateral direction (“L”). Other degrees of freedom are possible, such as rotation about one or more other axes, such as rotation about a transverse axis (indicated as “R”).

The coordinate system (including axes A, T, and L) shown in FIG. 10A can have an origin located at an isocenter 1010. The isocenter 1010 can be defined as a location where the radiation therapy beam 1008 intersects the origin of a coordinate axis, such as to deliver a prescribed radiation dose to a location on or within a patient. For example, the isocenter 1010 can be defined as a location where the radiation therapy beam 1008 intersects the patient for various rotational positions of the radiation therapy output 1004 as positioned by the gantry 1006 around the axis A.

In an example, a detector 1014 can be located within a field of the therapy beam 1008, such as can include a flat panel detector (e.g., a direct detector or a scintillation-based detector). The detector 1014 can be mounted on the gantry 1006 opposite the radiation therapy output 1004, such as to maintain alignment with the therapy beam 1008 as the gantry 1006 rotates. In this manner, the detector 1014 can be used to monitor the therapy beam 1008 or the detector 1014 can be used for imaging, such as portal imaging.

In an illustrative example, one or more of the platform 1016, the therapy output 1004, or the gantry 1006 can be automatically positioned, and the therapy output 1004 can establish the therapy beam 1008 according to a specified dose for a particular therapy delivery instance. A sequence of therapy deliveries can be specified according to a radiation therapy treatment plan, such as using one or more different orientations or locations of the gantry 1006, platform 1016, or therapy output 1004. The therapy deliveries can occur sequentially, but can intersect in a desired therapy locus on or within the patient, such as at the isocenter 1010. A prescribed cumulative dose of radiation therapy can thereby be delivered to the therapy locus while damage to tissue nearby the therapy locus is reduced or avoided.

As mentioned in relation to other examples herein, the radiation therapy system 1002 can include or can be coupled to an imaging acquisition system, such as to provide one or more of nuclear magnetic resonance (MR) imaging, X-ray imaging, such as can include computed tomography (CT) imaging, or ultrasound imaging. In an example, MR imaging information or other imaging information can be used to generate imaging information or visualizations equivalent to CT imaging, without requiring actual CT imaging. Such imaging can be referred to as “pseudo-CT” imaging.

FIG. 10B illustrates generally another example of a radiation therapy system 1002 (e.g., Leksell Gamma Knife manufactured by Elekta, AB, Stockholm, Sweden). As shown in FIG. 10B, in a radiation therapy treatment session, a patient 1052 can wear a coordinate frame 1054 to stabilize a portion of the patient's anatomy (e.g., the head) undergoing surgery or radiation therapy. Coordinate frame 1054 and a patient positioning system 1056 can establish a spatial coordinate system, which can be used while imaging a patient or during radiation surgery.

Radiation therapy system 1002 can include a protective housing 1064 to enclose a plurality of radiation sources 1062. Radiation sources 1062 can generate a plurality of radiation beams (e.g., beamlets) through beam channels 1066. The plurality of radiation beams can be included such as to focus on an isocenter 1010 from different directions. While each individual radiation beam can have a relatively low intensity, isocenter 1010 can receive a relatively high level of radiation when multiple doses from different radiation beams accumulate at isocenter 1010. In certain examples, isocenter 1010 can correspond to a target under surgery or treatment, such as a tumor.

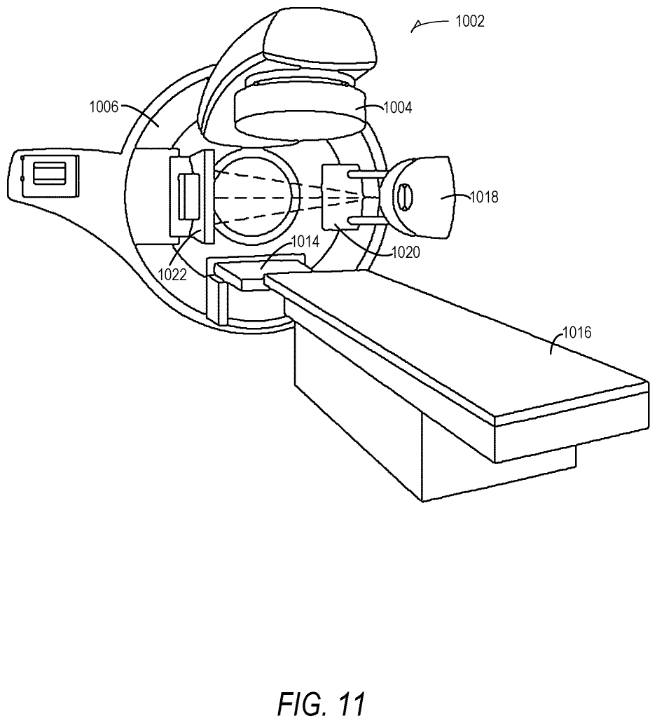

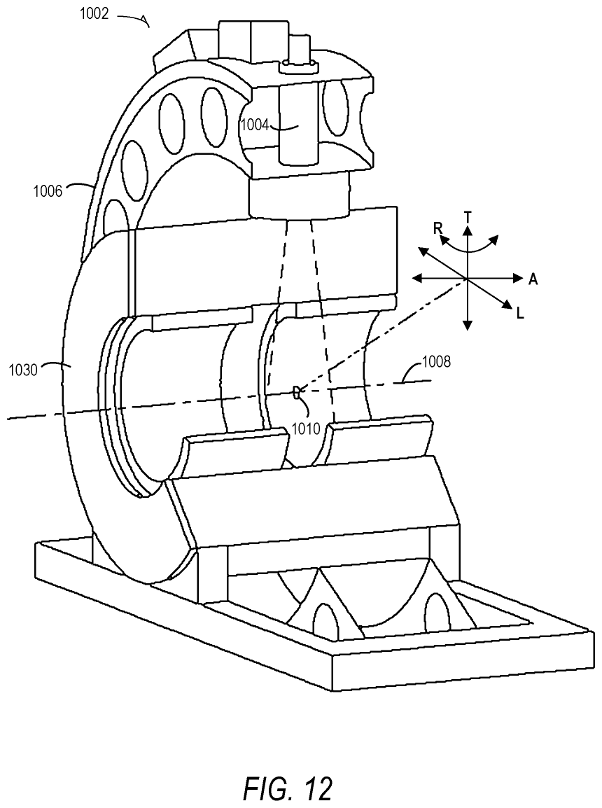

FIG. 11 illustrates generally an example of a system that can include a combined radiation therapy system 1002 and an imaging system, such as can include a computed tomography (CT) imaging system. The CT imaging system can include an imaging X-ray source 1018, such as providing X-ray energy in a kiloelectron-Volt (keV) energy range. The imaging X-ray source 1018 provides one or more of fan-shaped or conical beam 1020 directed to an imaging detector 1022, such as a flat panel detector. The radiation therapy system 1002 can be similar to the system 1002 described in relation to FIG. 10A, such as including a radiation therapy output 1004, a gantry 1006, a platform 1016, and another flat panel detector 1014. As in the examples of FIG. 10A, FIG. 10B, and FIG. 12, the radiation therapy system 1002 can be coupled to, or can include, a high-energy accelerator included such as to provide a therapeutic radiation beam. The X-ray source 1018 can provide a comparatively-lower-energy X-ray diagnostic beam, for imaging.

In the illustrative example of FIG. 11, the radiation therapy output 1004 and the X-ray source 1018 can be mounted on the same rotating gantry 1006, rotationally separated from each other by 90 degrees. In another example, two or more X-ray sources can be mounted along the circumference of the gantry 1006, such as each having its own detector arrangement to provide multiple angles of diagnostic imaging concurrently. Similarly, multiple radiation therapy outputs 1004 can be provided.

FIG. 12 illustrates generally a partially cut-away view of an example of a system that can include a combined radiation therapy system 1002 and an imaging system, such as can include a nuclear magnetic resonance (MR) imaging system 1030. The MR imaging system 1030 can be arranged to define a “bore” around an axis (“A”), and the radiation therapy system can include a radiation therapy output 1004, such as to provide a radiation therapy beam 1008 directed to an isocenter 1010 within the bore along the axis, A. The radiation therapy output 1004 can include a collimator 1024, such as to one or more of control, shape, or modulate radiation therapy beam 1008 to direct the beam 1008 to a therapy locus aligned with a desired target locus within a patient. The patient can be supported by a platform, such as a platform positionable along one or more of an axial direction, A, a lateral direction, L, or a transverse direction, T. One or more portions of the radiation therapy system 1002 can be mounted on a gantry 1006, such as to rotate the radiation therapy output 1004 about the axis A.

FIG. 10A, FIG. 10B, FIG. 11, and FIG. 12 illustrate generally examples including a configuration where a therapy output can be rotated around a central axis (e.g., an axis “A”). Other radiation therapy output configurations can be used. For example, a radiation therapy output can be mounted on a robotic arm or manipulator, such as having multiple degrees of freedom. In yet another example, the therapy output can be fixed, such as located in a region laterally separated from the patient, and a platform supporting the patient can be used to align a radiation therapy isocenter with a specified target locus within the patient.

FIG. 13 illustrates generally an example of a multi-leaf collimator (MLC) 1032, such as can be used to one or more of shape, direct, or modulate an intensity of a radiation therapy beam. In FIG. 13, leaves 1332A through 1332J can be automatically positioned to define an aperture approximating a tumor 1040 cross-section or projection. The leaves 1332A through 1332J can be made of a material specified to attenuate or block the radiation beam in regions other than the aperture, in accordance with the radiation treatment plan. For example, the leaves 1332A through 1332J can include metallic plates, such as comprising tungsten, with a long axis of the plates oriented perpendicular to a beam direction, and having ends oriented parallel to the beam direction (as shown in the plane of the illustration of FIG. 13). A “state” of the MLC 1032 can be adjusted adaptively during a course of radiation therapy, such as to establish a therapy beam that better approximates a shape or location of the tumor 1040 or other target locus, as compared to using a static collimator configuration or as compared to using an MLC 1032 configuration determined exclusively using an “offline” therapy planning technique. A radiation therapy technique including using the MLC 1032 to produce a specified radiation dose distribution to a tumor or to specific areas within a tumor can be referred to as Intensity Modulated Radiation Therapy (IMRT).

FIG. 14 illustrates an exemplary radiotherapy system 1400 adapted to perform radiotherapy plan processing operations using one or more of the approaches discussed herein. These radiotherapy plan processing operations are performed to enable the radiotherapy system 1400 to provide radiation therapy to a patient based on specific aspects of captured medical imaging data and therapy dose calculations or radiotherapy machine configuration parameters.

The radiotherapy system 1400 can include a radiotherapy processing computing system 1410 which hosts treatment processing logic 1420. Specifically, treatment processing logic 1420 implements various techniques discussed herein to detect object movement during real-time delivery of a radiotherapy session and to update a therapy protocol. It will be understood, however, that many variations and use cases of the following treatment processing logic 1420 can be provided, including in data verification, visualization, and other medical evaluative and diagnostic settings. The radiotherapy processing computing system 1410 can be connected to a network (not shown), and such network can be connected to the Internet. For instance, a network can connect the radiotherapy processing computing system 1410 with one or more medical information sources (e.g., a radiology information system (RIS), a medical record system (e.g., an electronic medical record (EMR)/electronic health record (EHR) system), an oncology information system (OIS)), one or more image data sources 1450, an image acquisition device 1470 (e.g., an imaging modality), a treatment device 1480 (e.g., a radiation therapy device, also referred to herein as a radiotherapy device), and treatment data source(s) 1460.

As an example, the radiotherapy processing computing system 1410 can be included such as to receive, at a first time point during the given radiation session, a first imaging slice comprising an object depicted in a 3D pre-treatment volumetric image. The first imaging slice is defined along a first plane (e.g., sagittal plane). At the first time point during the given radiation session, a second imaging slice is accessed comprising the object that was obtained at a second time point that precedes the first time point during the given radiation session. The second imaging slice is defined along a second plane (e.g., coronal plane). The system determines movement of the object at the first time point along first and second directions (e.g., along the sagittal plane and left/right sides) based on the registering the first imaging slice against the volumetric image. Movement of the object along a third direction (e.g., the coronal plane) is determined at the first time point based on the second imaging slice.

Specifically, the radiotherapy processing computing system 1410 can determine movement of an object according to a second technique, during a radiotherapy treatment session, along three directions (e.g., sagittal, coronal, and left/right planes). It does so by registering an image (e.g., a cine image) received along one plane (e.g., the sagittal plane), at a particular point in the treatment session, with a pre-treatment 3D volumetric image. The result provides movement of the object along the sagittal plane and the left/right plane. At the same particular point, the radiotherapy processing computing system 1410 retrieves a previously obtained image (e.g., a cine image) along the other plane (e.g., the coronal plane). The radiotherapy processing computing system 1410 uses the position of the object in the previously obtained image slice to determine movement of the object along the coronal plane. As a result, movement of the object can be determined at a given time point in the treatment session along three directions (e.g., 3D movement of the object can be determined). For example, the first direction corresponds to an anterior/posterior direction, the second direction corresponds to a superior/inferior direction, and the third direction corresponds to a left/right direction. The object can include at least one of an anatomy portion of a patient, a pixel, or any defined image portion. The first and second imaging slices are planar or orthogonal but can be any two imaging slices along any other parallel or perpendicular direction.

In some cases, in order to determine movement of the object along the current plane direction (e.g., the direction corresponding to the image that is currently obtained rather than the previously obtained image), the radiotherapy processing computing system 1410 extracts, from the volumetric image, a first two-dimensional (2D) slice along the first plane. The radiotherapy processing computing system 1410 spatially registers the first imaging slice and the first 2D slice (e.g., by moving a portion of the object in the first imaging slice to register the object in the first imaging slice with a portion of the object in the first 2D slice.

In an example, the radiotherapy processing computing system 1410 registers the second imaging slice (corresponding to an earlier time point in the treatment session) against the pre-treatment image to determine movement along the third direction. In such cases, the radiotherapy processing computing system 1410 defines the first and second imaging slices (defined along respective first and second planes) as moving images and the 2D slice is extracted from the volumetric pre-treatment image as the fixed image to perform 2D/2D image registration. The radiotherapy processing computing system 1410 then determines movement along the three directions based on the registered first and second image slices with the 3D pre-treatment volumetric image.

In an example, radiotherapy processing computing system 1410 computes in-plane shifts of the object along the first and second directions based on the first imaging slice and the first 2D slice. The radiotherapy processing computing system 1410 computes an out-of-plane shift value along the third direction based on the second imaging slice. For example, the radiotherapy processing computing system 1410 computes in-plane shifts of the object along the sagittal direction and the left/right direction based on the first imaging slice that is defined along the sagittal direction and the first 2D slice extracted from the pre-treatment volumetric image. The radiotherapy processing computing system 1410 computes an out-of-plane shift value along the coronal direction based on the second imaging slice that is defined along the coronal direction.

In some cases, the radiotherapy processing computing system 1410 predicts a difference between the second imaging slice defined along the second plane and an expected imaging slice defined along the second plane at the first time point. For example, the radiotherapy processing computing system 1410 applies a machine learning technique to the second imaging slice (obtained at a prior point in time) to predict or estimate the second imaging slice at the current point in time in the radiotherapy session. The predicted or estimated second imaging slice defined along the second plane can then be used in conjunction with the first imaging slice defined along the first plane to determine movement of the object along three directions at the current time point.

In an example, the radiotherapy processing computing system 1410 determines a metric indicative of an alignment between a portion of the object from the first imaging slice and a portion of the object from the first 2D slice. The radiotherapy processing computing system 1410 adjusts a position of the second imaging slice to improve an alignment between the portion of the object from the first imaging slice and the portion of the object from the first 2D slice. In some cases, the metric is determined by masking a portion of the first imaging slice and a slice of the volumetric image. In some cases, the metric is determined by determining a similarity between a line of intersection between the portion of the object from the first imaging slice and the portion of the object from a slice of the volumetric image.

In an example, the radiotherapy processing computing system 1410 determines movement during a radiotherapy treatment session according to a third technique. For example, the radiotherapy processing computing system 1410 receives, at a first time point during a given radiation session, a first imaging slice comprising an object. The first imaging slice can correspond to a first plane (e.g., the sagittal plane). The radiotherapy processing computing system 1410 accesses, at the first time point during the given radiation session, a composite imaging slice corresponding to the first plane. The composite imaging slice is generated using a plurality of imaging slices that were obtained prior to the first time point. The radiotherapy processing computing system 1410 spatially registers the first imaging slice and the composite imaging slice to determine movement of the object. The radiotherapy processing computing system 1410 generates an updated therapy protocol to control delivery of a therapy beam based on the determined movement.

In some cases, the radiotherapy processing computing system 1410 registers the first imaging slice and the composite imaging slice by mask limiting pixels considered in the registration in one or both of the first imaging slice and the composite imaging slice. The composite imaging slice is registered to the two-dimensional slice extracted from the volumetric image before the first imaging slice is registered to the composite imaging slice. In some cases, the composite image that is registered to the two-dimensional slice extracted from the volumetric image is manually adjusted by a user input to specify or change a position or portion of the composite image that is registered relative to the two-dimensional slice.

In an example, the composite image is a first composite image. At a second time point during the given radiation session, the radiotherapy processing computing system 1410 receives a second imaging slice that corresponds to a second plane (e.g., a coronal plane). The radiotherapy processing computing system 1410 accesses, at the second time point during the given radiation session, a second composite imaging slice corresponding to the second plane. The second composite imaging slice is generated using another plurality of imaging slices obtained prior to the second time point. The radiotherapy processing computing system 1410 spatially registers the second imaging slice and the second composite imaging slice, including moving a portion of the object in the second imaging slice to register the object in the second imaging slice with a portion of the object in the second composite imaging slice.

In an example, the composite image is generated by averaging a selected subset of imaging slices. For example, a plurality of imaging slices are obtained prior to activating the radiotherapy beam during the given radiation session. A given one of the slices that corresponds to a reference breathing phase is selected as a target image. The radiotherapy processing computing system 1410 deformably registers a remaining set of the plurality of imaging slices to the selected target image. The radiotherapy processing computing system 1410 averages the deformably registered plurality of imaging slices and determines an average offset for each of the plurality of imaging slices. The radiotherapy processing computing system 1410 resamples the imaging slices based on the average offset to select a subset of the resampled imaging slices based on a distance of each of the resampled imaging slices to the target image (e.g., the selected imaging slice). For example, the radiotherapy processing computing system 1410 selects those imaging slices for which the distances between the slices and the target slice is less than a specified threshold. The composite image is then generated by averaging the selected subset of the resampled plurality of imaging slices.

In an example, the radiotherapy processing computing system 1410 determines movement during a radiotherapy treatment session according to a fourth technique. For example, the radiotherapy processing computing system 1410 receives, at a first time point in a given radiation session, a first imaging slice corresponding to a first plane (e.g., a 2D cine image corresponding to or defined along a sagittal plane). The radiotherapy processing computing system 1410 encodes the first imaging slice to a lower dimensional representation (e.g., by converting principal component analysis (PCA) components of a deformable vector field (DVF) of the first imaging slice to estimate the PCA components of the second imaging slice). In one example, the encoding the slices to lower dimensional representation can include reducing the amount of data used to represent a given slice by converting the standard used to represent the slice from one standard to another. The radiotherapy processing computing system 1410 applies a trained machine learning model (e.g., a support vector machine or random forest machine or neural network) to the encoded first imaging slice to estimate an encoded version of a second imaging slice corresponding to a second plane (e.g., the coronal plane) at the first time point to provide a pair of imaging slices for the first time point. The radiotherapy processing computing system 1410 simultaneously spatially registers the pair of imaging slices to a volumetric image (e.g., a 3D pre-treatment volume of the patient), received prior to the given radiation session, comprising a time-varying object to calculate displacement of the object. The radiotherapy processing computing system 1410 generates an updated therapy protocol to control delivery of a therapy beam based on the calculated displacement of the object. In some cases, prior to registering the images, the radiotherapy processing computing system 1410 decodes the estimated encoded version of the second imaging slice to obtain the second imaging slice at the first time point. For example, the radiotherapy processing computing system 1410 converts or upscales the data used to represent the imaging slice from one standard to another. In order to encode and decode the slices, according to the disclosed techniques, any suitable compression and decompression algorithm can be used.

In some cases, the pair of imaging slices include a stereo pair of imaging slices, such that the first plane is orthogonal to the second plane or the first plane is tilted (e.g., non-orthogonal) relative to the second plane. In some cases, to train the machine learning model to perform the prediction in the fourth technique, the radiotherapy processing computing system 1410 receives, prior to the given radiation session, a first sequence of training imaging slices corresponding to the first plane. The radiotherapy processing computing system 1410 also receives, prior to the given radiation session, a second sequence of training imaging slices corresponding to the second plane. The second sequence of training imaging slices can be received concurrently with or alternatively with the first sequence of imaging slices. The radiotherapy processing computing system 1410 encodes the first and second sequences to a lower dimensional representation (e.g., converts the data used to represent the first and second sequences from one standard to another to reduce the amount of data used to represent the sequences). In some implementations, the radiotherapy processing computing system 1410 interpolates a first training image slice of the first sequence of training imaging slices corresponding to a first training time point to generate a first interpolated training imaging slice corresponding to a second training time point when a second training image slice of the second sequence of training imaging slices is received at the second training time point. Specifically, the radiotherapy processing computing system 1410 interpolates or generates an expected version of the first training image slice for a next adjacent time point that corresponds to the time point at which one of the training imaging slices of the second plane is received. The radiotherapy processing computing system 1410 trains the machine learning model based on the interpolated image to predict a first image corresponding to the first plane from a second image corresponding to the second plane. The radiotherapy processing computing system 1410 continuously or periodically trains the machine learning model during the given radiation session as new imaging slices are captured and received.

The radiotherapy processing computing system 1410 can include processing circuitry 1412, memory 1414, a storage device 1416, and other hardware and software-operable features such as a user interface 1442, a communication interface (not shown), and the like. The storage device 1416 can store transitory or non-transitory computer-executable instructions, such as an operating system, radiation therapy treatment plans (e.g., training data, treatment planning strategies, patient movement models, patient deformation models, beam delivery segment information, 5D and/or 2D image information for a patient, and device adjustment parameters, and the like), software programs (e.g., image processing software, image or anatomical visualization software, etc.), and any other computer-executable instructions to be executed by the processing circuitry 1412.

In an example, the processing circuitry 1412 can include a processing device, such as one or more general-purpose processing devices such as a microprocessor, a central processing unit (CPU), a graphics processing unit (GPU), an accelerated processing unit (APU), or the like. More particularly, the processing circuitry 1412 can be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processing circuitry 1412 can also be implemented by one or more special-purpose processing devices such as an application-specific integrated circuit (ASIC), a field programmable gate array (FPGA), a digital signal processor (DSP), a System on a Chip (SoC), or the like.

As would be appreciated by those skilled in the art, in some examples, the processing circuitry 1412 can be a special-purpose processor, rather than a general-purpose processor. The processing circuitry 1412 can include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processing circuitry 1412 can also include graphical processing units such as a GPU from the GeForce®, Quadro®, Tesla® family manufactured by Nvidia™, GMA, Iris™ family manufactured by Intel™, or the Radeon™ family manufactured by AMD™. The processing circuitry 1412 can also include accelerated processing units such as the Xeon Phi™ family manufactured by Intel™. The disclosed examples are not limited to any type of processor(s) otherwise included such as to meet the computing demands of identifying, analyzing, maintaining, generating, and/or providing large amounts of data or manipulating such data to perform the methods disclosed herein. In addition, the term “processor” can include more than one physical (circuitry based) or software-based processor, for example, a multi-core design or a plurality of processors each having a multi-core design. The processing circuitry 1412 can execute sequences of transitory or non-transitory computer program instructions, stored in memory 1414, and accessed from the storage device 1416, to perform various operations, processes, methods that will be explained in greater detail below. It should be understood that any component in radiotherapy system 1400 can be implemented separately and operate as an independent device and can be coupled to any other component in radiotherapy system 1400 to perform the techniques described in this disclosure.

The memory 1414 can comprise read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a compact disc read-only memory (CD-ROM), a digital versatile disc (DVD) or other optical storage, a cassette tape, other magnetic storage device, or any other non-transitory medium that can be used to store information including images, training data, ML technique parameters, device adaptation functions, data, or transitory or non-transitory computer-executable instructions (e.g., stored in any format) capable of being accessed by the processing circuitry 1412, or any other type of computer device. For instance, the computer program instructions can be accessed by the processing circuitry 1412, read from the ROM, or any other suitable memory location, and loaded into the RAM for execution by the processing circuitry 1412.

The storage device 1416 can constitute a drive unit that can include a transitory or non-transitory machine-readable medium on which is stored one or more sets of transitory or non-transitory instructions and data structures (e.g., software) embodying or utilized by any one or more of the methodologies or functions described herein (including, in various examples, the treatment processing logic 1420 and the user interface 1442). The instructions can also reside, completely or at least partially, within the memory 1414 and/or within the processing circuitry 1412 during execution thereof by the radiotherapy processing computing system 1410, with the memory 1414 and the processing circuitry 1412 also constituting transitory or non-transitory machine-readable media.

The memory 1414 and the storage device 1416 can constitute a non-transitory computer-readable medium. For example, the memory 1414 and the storage device 1416 can store or load transitory or non-transitory instructions for one or more software applications on the computer-readable medium. Software applications stored or loaded with the memory 1414 and the storage device 1416 can include, for example, an operating system for common computer systems as well as for software-controlled devices. The radiotherapy processing computing system 1410 can also operate a variety of software programs comprising software code for implementing the treatment processing logic 1420 and the user interface 1442. Further, the memory 1414 and the storage device 1416 can store or load an entire software application, part of a software application, or code or data that is associated with a software application, which is executable by the processing circuitry 1412. In a further example, the memory 1414 and the storage device 1416 can store, load, and manipulate one or more radiation therapy treatment plans, imaging data, segmentation data, treatment visualizations, histograms or measurements, and the like. It is contemplated that software programs can be stored not only on the storage device 1416 and the memory 1414 but also on a removable computer medium, such as a hard drive, a computer disk, a CD-ROM, a DVD, a Blu-Ray DVD, USB flash drive, a SD card, a memory stick, or any other suitable medium; such software programs can also be communicated or received over a network.

The radiotherapy processing computing system 1410 can also include a communication interface, network interface card, and communications circuitry. An example communication interface can include, for example, a network adaptor, a cable connector, a serial connector, a USB connector, a parallel connector, a high-speed data transmission adaptor (e.g., such as fiber, USB 5.0, thunderbolt, and the like), a wireless network adaptor (e.g., such as a IEEE 402.11/Wi-Fi adapter), a telecommunication adapter (e.g., to communicate with 5G, 4G/LTE, and 5G, networks and the like), and the like. Such a communication interface can include one or more digital and/or analog communication devices that permit a machine to communicate with other machines and devices, such as remotely located components, via a network. The network can provide the functionality of a local area network (LAN), a wireless network, a cloud computing environment (e.g., software as a service, platform as a service, infrastructure as a service, etc.), a client-server, a wide area network (WAN), and the like. For example, the network can be a LAN or a WAN that can include other systems (including additional image processing computing systems or image-based components associated with medical imaging or radiotherapy operations).

In an example, the radiotherapy processing computing system 1410 can obtain image data 1452 from the image data source 1450 (e.g., CT, PET, and/or MR images), for hosting on the storage device 1416 and the memory 1414. In yet another example, the software programs can substitute functions of the patient images such as signed distance functions or processed versions of the images that emphasize some aspect of the image information.

In an example, the radiotherapy processing computing system 1410 can obtain or communicate image data 1452 from or to image data source 1450. In further examples, the treatment data source 1460 receives or updates the planning data 1462 as a result of radiotherapy device parameter adjustments or segment adaptation or detecting movement of an object (e.g., a target locus).

The processing circuitry 1412 can be communicatively coupled to the memory 1414 and the storage device 1416, and the processing circuitry 1412 can be included such as to execute computer-executable instructions stored thereon from either the memory 1414 or the storage device 1416. The processing circuitry 1412 can execute instructions to cause medical images from the image data 1452 to be received or obtained in memory 1414 and processed using the treatment processing logic 1420.

In addition, the processing circuitry 1412 can utilize software programs to generate intermediate data such as updated parameters to be used, for example, by a neural network model, a deep learning neural network model, a regression technique comprising a support vector machine or random forest, machine learning model, or other aspects involved with generation of device parameter adjustments or segment adaptation, as discussed herein. Further, such software programs can utilize the treatment processing logic 1420 to produce updated radiotherapy parameters to provide to the treatment data source 1460 to modify a dose delivered to a target within a given fraction and/or for presentation on output device 1446, using the techniques further discussed herein. The processing circuitry 1412 can subsequently then transmit the updated radiotherapy parameters via a communication interface and the network to the treatment device 1480, where the updated parameters will be used to treat a patient with radiation via the treatment device 1480. Radiotherapy parameters (also referred to as control points) can include, for each segment or portion of a given treatment fraction, MLC positions and settings, gantry angle, radiation dose amount (e.g., amount of monitor units (MU)), radiotherapy beam direction, radiation beam size, arc placement, beam on and off time duration, machine parameters, gantry speed, MRI pulse sequence, any combination thereof, and so forth.

In an example, the image data 1452 can include one or more MR images (e.g., 2D MRI, 5D MRI, 2D streaming MRI, 4D MRI, 4D volumetric MRI, 4D cine MRI, etc.), functional MRI images (e.g., fMRI, DCE-MRI, diffusion MRI), Computed Tomography (CT) images (e.g., 2D CT, 2D Cone beam CT, 5D CT, 5D CBCT, 4D CT, 4DCBCT), ultrasound images (e.g., 2D ultrasound, 5D ultrasound, 4D ultrasound), Positron Emission Tomography (PET) images, X-ray images, fluoroscopic images, radiotherapy portal images, Single-Photo Emission Computed Tomography (SPECT) images, computer-generated synthetic images (e.g., pseudo-CT images), radio-beacons, laser scanning of the patient surface, and the like. Further, the image data 1452 can also include or be associated with medical image processing data, for instance, training images, ground truth images, contoured images, and dose images. In other examples, an equivalent representation of an anatomical area can be represented in non-image formats (e.g., coordinates, mappings, etc.).

In an example, the image data 1452 can be received from the image acquisition device 1470 and stored in one or more of the image data sources 1450 (e.g., a Picture Archiving and Communication System (PACS), a Vendor Neutral Archive (VNA), a medical record or information system, a data warehouse, etc.). Accordingly, the image acquisition device 1470 can comprise an MRI imaging device, a CT imaging device, a PET imaging device, an ultrasound imaging device, a fluoroscopic device, a SPECT imaging device, an integrated Linear Accelerator and MRI imaging device, CBCT imaging device, or other medical imaging devices for obtaining the medical images of the patient. The image data 1452 can be received and stored in any type of data or any type of format (e.g., in a Digital Imaging and Communications in Medicine (DICOM) format) that the image acquisition device 1470 and the radiotherapy processing computing system 1410 can use to perform operations consistent with the disclosed examples. Further, in some examples, the models discussed herein can be trained to process the original image data format or a derivation thereof.

In an example, the image acquisition device 1470 can be integrated with the treatment device 1480 as a single apparatus (e.g., an MRI device combined with a linear accelerator, also referred to as an “MRI-Linac”). Such an MRI-Linac can be used, for example, to determine a location of a target in the patient, so as to direct the radiation beam accurately according to the radiation therapy treatment plan to a predetermined target. For instance, a radiation therapy treatment plan can provide information about a particular radiation dose to be applied to each patient. The radiation therapy treatment plan can also include other radiotherapy information and radiotherapy device parameters, such as beam angles, dose-volume-histogram information, the number of radiation beams to be used during therapy, the dose per beam, and the like. The MRI-Linac can be used to compute, generate, and/or update a patient deformation model to deform image portions of a 5D or 2D image of a patient corresponding to a given beam delivery segment. The MRI-Linac can be used to provide real-time patient images (or subsets of patient images) and machine settings or parameters at various increments/intervals of a treatment fraction to continuously or periodically compute dose for the increments/intervals and determine a real-time dose accumulation based on such computed doses.

The radiotherapy processing computing system 1410 can communicate with an external database through a network to send/receive a plurality of various types of data related to image processing and radiotherapy operations. For example, an external database can include machine data (including device constraints) that provides information associated with the treatment device 1480, the image acquisition device 1470, or other machines relevant to radiotherapy or medical procedures. Machine data information can include radiation beam size, arc placement, beam on and off time duration, machine parameters, segments, MLC configuration, gantry speed, MRI pulse sequence, and the like. The external database can be a storage device and can be equipped with appropriate database administration software programs. Further, such databases or data sources can include a plurality of devices or systems located either in a central or a distributed manner.

The radiotherapy processing computing system 1410 can collect and obtain data, and communicate with other systems, via a network using one or more communication interfaces, which are communicatively coupled to the processing circuitry 1412 and the memory 1414. For instance, a communication interface can provide communication connections between the radiotherapy processing computing system 1410 and radiotherapy system components (e.g., permitting the exchange of data with external devices). For instance, the communication interface can, in some examples, have appropriate interfacing circuitry from an output device 1446 or an input device 1448 to connect to the user interface 1442, which can be a hardware keyboard, a keypad, or a touch screen through which a user can input information into the radiotherapy system.

As an example, the output device 1446 can include a display device that outputs a representation of the user interface 1442 and one or more aspects, visualizations, or representations of the medical images, the treatment plans, and statuses of training, generation, verification, or implementation of such plans. The output device 1446 can include one or more display screens that display medical images, interface information, treatment planning parameters (e.g., contours, dosages, beam angles, labels, maps, etc.), treatment plans, image portions that are identified and deformed for a given treatment segment, a target, localizing a target and/or tracking a target, or any related information to the user. The output device 1446 can provide to a user visualization of movement of the target object or therapy locus during delivery of the radiotherapy treatment fraction.

The input device 1448 connected to the user interface 1442 can be a keyboard, a keypad, a touch screen or any type of device using which a user can input information to the radiotherapy system 1400. Alternatively, the output device 1446, the input device 1448, and features of the user interface 1442 can be integrated into a single device such as a smartphone or tablet computer (e.g., Apple iPad®, Lenovo Thinkpad®, Samsung Galaxy®, etc.).

Furthermore, any and all components of the radiotherapy system 1400 can be implemented as a virtual machine (e.g., via VMWare, Hyper-V, and the like virtualization platforms) or independent devices. For instance, a virtual machine can be software that functions as hardware. Therefore, a virtual machine can include at least one or more virtual processors, one or more virtual memories, and one or more virtual communication interfaces that together function as hardware. For example, the radiotherapy processing computing system 1410, the image data sources 1450, or like components, can be implemented as a virtual machine or within a cloud-based virtualization environment.

The image acquisition device 1470 can be included such as to acquire one or more images of the patient's anatomy for a region of interest (e.g., a target organ, a target tumor or both). Each image, typically a 2D image or slice, can include one or more parameters (e.g., a 2D slice thickness, an orientation, and a location, etc.). In an example, the image acquisition device 1470 can acquire a 2D slice in any orientation. For example, an orientation of the 2D slice can include a sagittal orientation, a coronal orientation, or an axial orientation. The processing circuitry 1412 can adjust one or more parameters, such as the thickness and/or orientation of the 2D slice, to include the target organ and/or target tumor. In an example, 2D slices can be determined from information such as a 5D CBCT or CT, or MRI volume. Such 2D slices can be acquired by the image acquisition device 1470 in “near real-time” while a patient is undergoing radiation therapy treatment, for example, when using the treatment device 1480 (with “near real-time” meaning acquiring the data without (or with minimal) lag between image acquisition and treatment, as known in the art). In an example, 5D volumetric representation of a region of interest can be generated using a stack of one or more 2D slices.

df

Example 1 is a computing device for motion tracking during radiation treatment of a patient, the computing device including a processor and a memory device, the memory device including instructions that, when executed by the processor, cause the computing device to: receive, during a radiation session, a first image set corresponding to a specified anatomical structure of the patient, the first image set of the specified anatomical structure captured at different times at a first plane; receive, during the radiation session, a second image set corresponding to the specified anatomical structure of the patient, the second image set of the specified anatomical structure captured at different times at a second plane oriented at an angle to the first plane; identify a first initial trajectory based on an interpolation of respective registration points of the specified anatomical structure for first and second image slices of the first image set, the first initial trajectory having an in-plane direction with at least one of the first and second planes; identify a second initial trajectory based on an interpolation of respective registration points of the specified anatomical structure for a first and second image slices of the second image set, the second initial trajectory in an in-plane direction with at least one of the first and second planes; determine, based on the first and second initial trajectories, a third initial trajectory including a through-plane coordinate for the first plane and a through-plane coordinate for the second plane; train a model, using training data obtained during the radiation session, the training data including the first, second, and third trajectories, to estimate a three dimensional (3D) position of the specified anatomical structure, the 3D position including a coordinate in a through-plane direction based on the respective through-plane coordinates corresponding with the first and second planes; output the model for use in generating an updated therapy protocol; and generating, using the model, an updated therapy protocol to control delivery of a beam of radiation.

In Example 2, the subject matter of Example 1 includes, wherein the memory device includes instructions that, when executed by the processor, cause the computing device to: determine movement of the specified anatomical structure, relative to at least one aperture, using the trained model; and generate the updated therapy protocol to control delivery of the beam based on the movement.

In Example 3, the subject matter of Examples 1-2 includes, wherein the memory device includes instructions that, when executed by the processor, cause the computing device to: calculate an interpolation between at least one initialization image and a template, the at least one initialization point obtained during a preparation session performed before the radiation session; wherein the instructions to identify the first and second trajectories include instructions to identify the interpolation between the at least one initialization point and the template.

In Example 4, the subject matter of Examples 1-3 includes, wherein the memory device includes instructions that, when executed by the processor, cause the computing device to: determine an error between one of the first or second trajectories and a predicted trajectory output by the model; and retrain the model based on the determined error.

In Example 5, the subject matter of Examples 1-4 includes, wherein instructions to train the model include instructions to regress the first trajectory, the second trajectory, and the third trajectory.

In Example 6, the subject matter of Examples 1-5 includes, wherein the memory device includes instructions that, when executed by the processor, cause the computing device to: determine movement, based on the model, in an in-plane direction at a latency equal to a latency for movement determined in the through-plane direction; and control delivery of a therapy beam at symmetrical margins for the in-plane direction and the through-plane direction.

In Example 7, the subject matter of Examples 1-6 includes, wherein instructions to a determine movement of the specified anatomical structure include instructions to offset movement of an aperture of a radiation source relative to the specified anatomical structure, the offset identified using the trained model.

Example 8 is a computer-implemented method for motion tracking during radiation treatment of a patient, the method comprising: receiving, during a radiation session, a first image set corresponding to a specified anatomical structure of the patient, the first image set of the specified anatomical structure captured at different times at a first plane; receiving, during the radiation session, a second image set corresponding to the specified anatomical structure of the patient, the second image set of the specified anatomical structure captured at different times at a second plane oriented at an angle to the first plane; identifying a first initial trajectory based on an interpolation of respective registration points of the specified anatomical structure for first and second image slices of the first image set, the first initial trajectory having an in-plane direction with at least one of the first and second planes; identifying a second initial trajectory based on an interpolation of respective registration points of the specified anatomical structure for first and second image slices of the second image set, the second initial trajectory in an inplane direction with at least one of the first and second planes; determining, based on the first and second initial trajectories, a third initial trajectory including a through-plane coordinate for the first plane and a through-plane coordinate for the second plane; training a model, using training data obtained during the radiation session, the training data including the first, second, and third trajectories, to estimate a three dimensional (3D) position of the specified anatomical structure, the 3D position including a coordinate in a through-plane direction based on the respective through-plane coordinates corresponding with the first and second planes; and outputting the model for use in generating an updated therapy protocol.

In Example 9, the subject matter of Example 8 includes, determining movement of the specified anatomical structure, relative to at least one aperture, using the trained model; and generating an updated therapy protocol to control delivery of a beam based on the movement.

In Example 10, the subject matter of Examples 8-9 includes, calculating an interpolation between at least one initialization image and a template, the at least one initialization point obtained during a preparation session performed before the radiation session; wherein identifying the first and second trajectories is based on the interpolation between the at least one initialization point and the template.

In Example 11, the subject matter of Examples 8-10 includes, determining an error between one of the first or second trajectories and a predicted trajectory output by the model; and retraining the model based on the determined error.

In Example 12, the subject matter of Examples 8-11 includes, wherein training the model includes regressing the first trajectory, the second trajectory, and the third trajectory.

In Example 13, the subject matter of Examples 8-12 includes, determining movement, based on the model, in an in-plane direction at a latency equal to a latency for movement determined in the through-plane direction; and controlling delivery of a therapy beam at symmetrical margins for the in-plane direction and the through-plane direction.

In Example 14, the subject matter of Examples 8-13 includes, wherein determining movement of the specified anatomical structure includes offsetting movement of an aperture of a radiation source relative to the specified anatomical structure, the offsetting identified using the trained model.

Example 15 is a method for motion tracking during radiation treatment of a patient, the method comprising: receiving, during a radiation session, a first image set corresponding to a specified anatomical structure of the patient, the first image set of the specified anatomical structure captured at different times at a first plane; identifying a first initial trajectory based on an interpolation of respective registration points of the specified anatomical structure for first and second image slices of the first image set, the first initial trajectory having an in-plane direction with the first plane; determining, based on the first initial trajectory, a through-plane initial trajectory including a through-plane coordinate for the first plane and a through-plane coordinate for the second plane; estimating, using the first initial trajectory and the through-plane initial trajectory as an input to a trained model, a three dimensional (3D) position of the specified anatomical structure, the 3D position including a coordinate in a through-plane direction based on the respective through-plane coordinates corresponding with the first and second planes; generating an updated therapy protocol based on predicted movement of the specified anatomical structure; and establishing or adjusting, based on the predicted movement, a shape of a radiotherapy beam delivered to the specified anatomical structure.

In Example 16, the subject matter of Example 15 includes, receiving, during the radiation session, a second image set corresponding to the specified anatomical structure of the patient, the second image set of the specified anatomical structure captured at different times at a second plane oriented at an angle to the first plane; identifying a second initial trajectory based on an interpolation of respective registration points of the specified anatomical structure for first and second images slices of the second image set, the second initial trajectory in an inplane direction with at least one of the first and second planes; and using the second initial trajectory as an input to the trained model to predict movement of the specified anatomical structure.

In Example 17, the subject matter of Example 16 includes, calculating an interpolation between at least one initialization image and a template, the at least one initialization point obtained during a preparation session performed before the radiation session; wherein identifying the first and second trajectories is based on the interpolation between the at least one initialization point and the template.

In Example 18, the subject matter of Examples 15-17 includes, determining movement, using on the model, in an in-plane direction at a latency equal to a latency for movement determined in the through-plane direction; and controlling delivery of a therapy beam at symmetrical margins for the in-plane direction and the through-plane direction.

In Example 19, the subject matter of Example 18 includes, wherein determining movement of the specified anatomical structure includes offsetting movement of an aperture of a radiation source relative to the specified anatomical structure, the offsetting identified using the trained model.

Example 20 is at least one machine-readable medium including instructions that, when executed by processing circuitry, cause the processing circuitry to perform operations to implement of any of Examples 1-19.

Example 21 is an apparatus comprising means to implement of any of Examples 1-19.

Example 22 is a system to implement of any of Examples 1-19.

Example 23 is a method to implement of any of Examples 1-19.

Each of the non-limiting examples described in this document can stand on its own, or can be combined in various permutations or combinations with one or more of the other examples.

The above detailed description can include references to the accompanying drawings, which form a part of the detailed description. The drawings show, by way of illustration, specific examples in which the inventive subject matter can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the inventive subject matter should be determined with reference to the appended claims, along with the full scope of equivalents to which such claims are entitled.

1. A computing device for motion tracking during radiation treatment of a patient, the computing device including a processor and a memory device, the memory device including instructions that, when executed by the processor, cause the computing device to:
receive, during a radiation session, a first image set corresponding to a specified anatomical structure of the patient, the first image set of the specified anatomical structure captured at different times at a first plane;
receive, during the radiation session, a second image set corresponding to the specified anatomical structure of the patient, the second image set of the specified anatomical structure captured at different times at a second plane oriented at an angle to the first plane;
identify a first initial trajectory based on an interpolation of respective registration points of the specified anatomical structure for first and second image slices of the first image set, the first initial trajectory having an in-plane direction with at least one of the first and second planes;
identify a second initial trajectory based on an interpolation of respective registration points of the specified anatomical structure for a first and second image slices of the second image set, the second initial trajectory in an in-plane direction with at least one of the first and second planes;
determine, based on the first and second initial trajectories, a third initial trajectory including a through-plane coordinate corresponding with the first plane and a through-plane coordinate corresponding with the second plane;
train a model, using training data obtained during the radiation session, the training data including the first, second, and third trajectories, to estimate a three dimensional (3D) position of the specified anatomical structure, the 3D position including a coordinate in a through-plane direction based on the respective through-plane coordinates corresponding with the first and second planes;
output the model for use in generating an updated therapy protocol; and
generate, using the model, an updated therapy protocol to control delivery of a beam of radiation.

2. The computing device of claim 1, wherein the memory device includes instructions that, when executed by the processor, cause the computing device to:
determine movement of the specified anatomical structure, relative to at least one aperture, using the trained model; and
generate the updated therapy protocol to control delivery of the beam based on the movement.

3. The computing device of claim 1, wherein the memory device includes instructions that, when executed by the processor, cause the computing device to:
calculate an interpolation between at least one initialization image and a template, the at least one initialization point obtained during a preparation session performed before the radiation session;
wherein the instructions to identify the first and second trajectories include instructions to identify the interpolation between the at least one initialization point and the template.

4. The computing device of claim 1, wherein the memory device includes instructions that, when executed by the processor, cause the computing device to:
determine an error between one of the first or second trajectories and a predicted trajectory output by the model; and
retrain the model based on the determined error.

5. The computing device of any of claim 1, wherein instructions to train the model include instructions to regress the first trajectory, the second trajectory, and the third trajectory.

6. The computing device of any of claim 1, wherein the memory device includes instructions that, when executed by the processor, cause the computing device to:
determine movement, based on the model, in an in-plane direction at a latency equal to a latency for movement determined in the through-plane direction; and
control delivery of a therapy beam at symmetrical margins for the in-plane direction and the through-plane direction.

7. The computing device of claim 1, wherein instructions to determine movement of the specified anatomical structure include instructions to offset movement of an aperture of a radiation source relative to the specified anatomical structure, the offset identified using the trained model.

8. A computer-implemented method for motion tracking during radiation treatment of a patient, the method comprising:
receiving, during a radiation session, a first image set corresponding to a specified anatomical structure of the patient, the first image set of the specified anatomical structure captured at different times at a first plane;
receiving, during the radiation session, a second image set corresponding to the specified anatomical structure of the patient, the second image set of the specified anatomical structure captured at different times at a second plane oriented at an angle to the first plane;
identifying a first initial trajectory based on an interpolation of respective registration points of the specified anatomical structure for first and second image slices of the first image set, the first initial trajectory having an in-plane direction with at least one of the first and second planes;
identifying a second initial trajectory based on an interpolation of respective registration points of the specified anatomical structure for first and second image slices of the second image set, the second initial trajectory in an inplane direction with at least one of the first and second planes;
determining, based on the first and second initial trajectories, a third initial trajectory including a through-plane coordinate corresponding with the first plane and a through-plane coordinate corresponding with the second plane;
training a model, using training data obtained during the radiation session, the training data including the first, second, and third trajectories, to estimate a three dimensional (3D) position of the specified anatomical structure, the 3D position including a coordinate in a through-plane direction based on the respective through-plane coordinates corresponding with the first and second planes; and
outputting the model for use in generating an updated therapy protocol.

9. The method of claim 8, further comprising:
determining movement of the specified anatomical structure, relative to at least one aperture, using the trained model; and
generating an updated therapy protocol to control delivery of a beam based on the movement.

10. The method of claim 8, further comprising:
calculating an interpolation between at least one initialization image and a template, the at least one initialization point obtained during a preparation session performed before the radiation session;
wherein identifying the first and second trajectories is based on the interpolation between the at least one initialization point and the template.

11. The method of claim 8, further comprising:
determining an error between one of the first or second trajectories and a predicted trajectory output by the model; and
retraining the model based on the determined error.

12. The method of claim 8, wherein training the model includes regressing the first trajectory, the second trajectory, and the third trajectory.

13. The method of claim 8, further comprising:
determining movement, based on the model, in an in-plane direction at a latency equal to a latency for movement determined in the through-plane direction; and
controlling delivery of a therapy beam at symmetrical margins for the in-plane direction and the through-plane direction.

14. The method of claim 8, wherein determining movement of the specified anatomical structure includes offsetting movement of an aperture of a radiation source relative to the specified anatomical structure, the offsetting identified using the trained model.

15. A method for motion tracking during radiation treatment of a patient, the method comprising:
receiving, during a radiation session, a first image set corresponding to a specified anatomical structure of the patient, the first image set of the specified anatomical structure captured at different times at a first plane;
identifying a first initial trajectory based on an interpolation of respective registration points of the specified anatomical structure for first and second image slices of the first image set, the first initial trajectory having an in-plane direction with the first plane;
determining, based on the first initial trajectory, a through-plane initial trajectory including a through-plane coordinate corresponding with the first plane and a through-plane coordinate corresponding with the second plane;
estimating, using the first initial trajectory and the through-plane initial trajectory as an input to a trained model, a three dimensional (3D) position of the specified anatomical structure, the 3D position including a coordinate in a through-plane direction based on the respective through-plane coordinates corresponding with the first and second planes;
generating an updated therapy protocol based on predicted movement of the specified anatomical structure; and
establishing or adjusting, based on the predicted movement, a shape of a radiotherapy beam delivered to the specified anatomical structure.

16. The method of claim 15, further comprising:
receiving, during the radiation session, a second image set corresponding to the specified anatomical structure of the patient, the second image set of the specified anatomical structure captured at different times at a second plane oriented at an angle to the first plane;
identifying a second initial trajectory based on an interpolation of respective registration points of the specified anatomical structure for first and second images slices of the second image set, the second initial trajectory in an inplane direction with at least one of the first and second planes; and
using the second initial trajectory as an input to the trained model to predict movement of the specified anatomical structure.

17. The method of claim 16, further comprising:
calculating an interpolation between at least one initialization image and a template, the at least one initialization point obtained during a preparation session performed before the radiation session;
wherein identifying the first and second trajectories is based on the interpolation between the at least one initialization point and the template.

18. The method of any of claim 15, further comprising:
determining movement, using on the model, in an in-plane direction at a latency equal to a latency for movement determined in the through-plane direction; and
controlling delivery of a therapy beam at symmetrical margins for the in-plane direction and the through-plane direction.

19. The method of claim 18, wherein determining movement of the specified anatomical structure includes offsetting movement of an aperture of a radiation source relative to the specified anatomical structure, the offsetting identified using the trained model.
